(19) United States
(12) Patent Application Publication
(10) Pub. No.: US 2021/0012865 A1
(43) Pub. Date: Jan. 14, 2021

(54) METHODS AND SYSTEMS FOR BIOMARKER ANALYSIS

(71) Applicant: SRI INTERNATIONAL, Menlo Park, CA (US)

(72) Inventors: Harold Stanley Javitz, Menlo Park, CA (US); David Cooper, Palo Alto, CA (US); Michael Greenstein, Los Altos, CA (US); Shirley Lee, Newark, CA (US)

(21) Appl. No.: 16/982,324

(22) PCT Filed: Mar. 19, 2019
(86) PCT No.: PCT/US2019/022913

§ 371 Date: Sep. 18, 2020

Related U.S. Application Data
(60) Provisional application No. 62/645,021, filed on Mar. 19, 2018.

(51) Int. Cl. G16H 10/40; G01N 33/573; G01N 33/68

(52) U.S. Cl. CPC G16H 10/40; G01N 33/6893; G01N 33/573

(57) ABSTRACT

Method and systems are described comprising receiving a plurality of human subject biomarker concentration values associated with a human subject, wherein the plurality of human subject biomarkers is associated with a condition, determining, based on the plurality of human subject biomarker concentration values, a human subject test statistic, comparing the human subject test statistic to a test statistic threshold, wherein the test statistic threshold is derived based on non-human primate (NHP) subject data, and, determining, based on the human subject test statistic exceeding the test statistic threshold, that the human subject has the condition.

### CROSS REFERENCE TO RELATED PATENT APPLICATION

This application claims priority to U.S. Provisional Application No. 62/645,021 entitled, “PERCENTILE ALGORITHM FOR COMBINING BIOMARKERS FOR WITHIN AND CROSS SPECIES ANALYSIS”, filed Mar. 19, 2018 herein incorporated by reference in its entirety.

### STATEMENT REGARDING FEDERALLY SPONSORED RESEARCH

This invention was made with Government support under HHSO100201000007C awarded by Biomedical Advanced Research and Development Authority, Department of Health and Human Services. The government has certain rights in the invention.

### BACKGROUND

A radiation biodosimeter that can be used at the point of need to triage individuals potentially exposed to ionizing radiation would have significant impact on the ability to provide timely and effective medical treatment and enable efficient use of scarce medical resources following a major nuclear event. Because there is limited data on the radiation response of healthy humans, and it is unethical to conduct such studies, no such device yet exists.

### SUMMARY

It is to be understood that both the following general description and the following detailed description are exemplary and explanatory only and are not restrictive.

A method is described comprising receiving a plurality of human subject biomarker concentration values associated with a human subject, wherein the plurality of human subject biomarkers is associated with a condition, determining, based on the plurality of human subject biomarker concentration values, a human subject test statistic, comparing the human subject test statistic to a test statistic threshold, wherein the test statistic threshold is derived in part based on non-human primate (NHP) subject data, and, determining, based on the human subject test statistic exceeding the test statistic threshold, that the human subject has the condition.

An apparatus is described comprising a housing comprising a port for receiving a test strip that supports lateral flow of a fluid sample along a lateral flow direction and comprises a plurality of zones wherein each of a plurality of human subject biomarkers is associated with one zone of the plurality of zones, and wherein a control is associated with at least one zone of the plurality of zones, wherein the plurality of human subject biomarkers are associated with a condition, a reader configured to obtain separable light intensity measurements from the plurality of zones, and a data analyzer configured to, convert, for each zone of the plurality of zones, a light intensity measurement into a human subject concentration value for the biomarker of the plurality of human subject biomarkers associated with a respective zone of the plurality of zones, determine, based on the plurality of human subject biomarker concentration values, a human subject test statistic, compare the human subject test statistic to a test statistic threshold, wherein the test statistic threshold is derived in part based on non-human primate (NHP) subject data, and determine, based on the human subject test statistic exceeding the test statistic threshold, that the human subject has the condition.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 shows an example process for determining whether a human subject has a condition based on an analysis of biomarker concentrations;

FIG. 2 shows an example process for determining whether a human subject has a condition based on an analysis of biomarker concentrations;

FIG. 3 shows an example process for determining parameters used in a process for determining whether a human subject has a condition based on an analysis of biomarker concentrations;

FIG. 4A shows an example lateral flow assay test strip;

FIG. 4B shows a fluid sample being applied to an application zone of the lateral flow assay test strip of FIG. 4A;

FIG. 4C shows the lateral flow assay test strip of FIG. 4B after the fluid sample has flowed across the test strip to an absorption zone;

FIG. 5 shows an example diagnostic test system configured for performing the disclosed methods;

FIG. 6 shows an example process for determining whether a human subject has a condition based on an analysis of biomarker concentrations;

FIG. 7 shows a block diagram of an operating environment for implementing the described methods;

FIG. 8 shows a heatmap showing the log 10 of the t-test p-values for each protein for each dose group and time point for the M918 study;

FIG. 9 shows boxplots from the M918 immunoassay data for the proteins AMY1A, FLT3L, AACT, and IL15;

FIG. 10 shows boxplots for NHP M073 (LBERI Cohort 1, left) and M103 (LBERI Cohort 2, right) data sets for AACT (top) and AMY1 (bottom);

FIG. 11 shows boxplots for NHP M073 (LBERI Cohort 1, left) and M103 (LBERI Cohort 2, right) data sets for FLT3L (top) and MCP1 (bottom);

FIG. 12 shows boxplots for NHP M073 (LBERI Cohort 1, left) and M103 (LBERI Cohort 2, right) data sets for NGAL;

FIG. 13 shows fold change plot for AACT, Flt3L, AMY1, NGAL, and MCP1 for NHPs (from studies M103 and M073);

FIG. 14 shows a Receiver Operating Characteristic (ROC) curve for all 894 NHP samples for the 3-marker panel (left) and 4-marker panel (right);

FIG. 15 shows cumulative distribution functions of the estimated probability of exposure for selected NHP subgroups;

FIG. 16 shows boxplots for human data sets for AACT, AMY1, Flt3L, IL15, MCP1, and NGAL. The horizontal scale for all plots in the log 10 of the measured plasma concentration in ng/ml;

FIG. 17 shows fold change plots for human TBI patients (top) and normal NHPs (bottom) for the four protein markers AMY1A, FLT3L, MCP1, and AACT for the case of identical fractionated dosing of 1.2 Gy administered 3× per day. Samples collected on Days 1, 2, and 3 were from subjects who received cumulative doses of 3.6, 7.2, and 10.8 Gy administered on the previous days;

FIG. 18 shows fold change plots for AMY1A, FLT3L, and MCP1 for NHPs for single acute doses of 3 Gy and 3.6 Gy and double and triple fractionated doses of 3 Gy and 3.6 Gy. The differences observed between fractionated and acute dosing on each day are not statistically significant;

FIG. 19 shows a ROC curve for all 1051 human normal, confounder group, and TBI patient samples. The vertical and horizontal lines demark 95% sensitivity and specificity. The total AUC is 0.96.

FIG. 20 shows cumulative distribution function (CDF) plots for human normals and post-exposure TBI patients for the proteins AMY1, FLT3L, and MCP1. The first three plots are for each individual protein. The last plot is the distribution obtained using all three proteins;

FIG. 21 shows a CDF plot of cumulative probability vs. the sum of −ln(p) across all of the biomarkers for human normals and TBI patients exposed to 3.6 and 7.2 Gy. The horizontal line is at the 95% percentile of the cumulative distribution and the vertical line intersects the x-axis at the threshold for predicting whether an observation is from and individual that was exposed to ˜2 Gy;

FIG. 22 shows CDFs for both unexposed humans and NHPs, as well as human TBI patients and healthy NHPs receiving a total fractionated dose of 3.6 Gy and healthy NHPs receiving single acute doses of 3 and 4 Gy;

FIG. 23 shows the distribution of the (PCA) test statistic for NHP both at baseline and at various radiation exposure levels; and

FIG. 24 shows shows the distribution of the PCA test statistic for NHP both at baseline and at an exposure of 4 Gy.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

In an aspect, methods and systems are described for using multiple biomarkers for within-species or cross-species analyses to classify samples as being from subjects having a condition or not having the condition. The condition may be, for example, exposure to radiation, sepsis, and the like. In an embodiment, samples may be classified as being from subjects that have been irradiated or not irradiated above a specified threshold. Parameters used to evaluate a sample in humans may be similar to those in Non-Human Primates (NHP) (other than normalization parameters such as the mean and standard deviation of the biomarkers in unexposed subjects). The methods and systems are insensitive to monotone transformations of the biomarkers and not dependent on an assumption of normality. The methods and systems are continuous, in that the methods and systems so not rely on thresholds for individual biomarkers. The classification methods described are derivable from non-irradiated samples. Irradiated samples may be used to determine which biomarkers to include in the classification methods and to estimate classification accuracy for irradiated samples. The methods are tunable so that the influence of individual biomarkers can be accentuated or depressed and so that the false positive rate for subpopulations with comorbidities can be adjusted to achieve a nominal alpha rate (e.g., risk of Type I error).

The methods and systems described transform observed values of each biomarker measured in an unknown sample into the corresponding percentile of that biomarker in the unirradiated sample distribution. Use of percentiles removes any assumption that the data are normally distributed and is insensitive to monotone transformations of the data. The percentiles are then transformed so that if the biomarker is overexpressed (underexpressed) in a sample from an irradiated subject, then the transformed percentile has a larger value when the biomarker is in the upper (or lower) percentile of the distribution of unirradiated observations. Use of a transformed percentile as the measure for each biomarker of its discrepancy from the unirradiated sample distribution removes the requirement of a simple threshold for each biomarker. This allows observations with biomarker values that are say, at the 94^{th} percentile to count almost as much as biomarker values at the 96^{th} percentile, increasing the sensitivity of the methods when combining across percentiles. A weighted sum of the transformed percentile values is calculated for each observation. A threshold for the sum of the transformed percentile values is then calculated so that the false positive rate for unirradiated observations is a pre-specified value (such as 5%). The methods do not depend on the use of data from irradiated samples. The methods are tunable by establishing different weights for the biomarkers in the final sum, and by truncating the contribution of any biomarker to the sum, and the weights and truncation can be subpopulation specific. In addition, different thresholds for the sum of the biomarker contributions can be established for different comorbid subpopulations to assure that the false positive rate for those subpopulations is set appropriately.

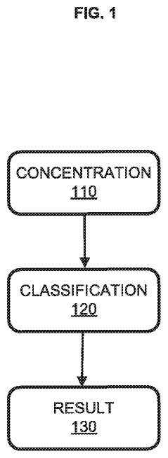

As shown in FIG. 1, the methods and systems described may comprise a concentration method 110 and a classification method 120. In an embodiment, some or all of the concentration method 110 and/or the classification method 120 may be embedded in an apparatus, such as a biodosimeter/analyzer. In an embodiment, some or all of the concentration method 110 and/or the classification method 120 may be performed by a computing device separate from the biodosimeter/analyzer. Some or all steps of the concentration method 110 and/or the classification method 120 may be performed by a processor of the biodosimeter/analyzer. The concentration method 110 and the classification method 120 may convert intensities of optical signals from test and control lines on linear flow assay (LFA) test strips, measured by a biodosimeter/analyzer, to a determination of whether an individual has been exposed to a radiation dose less than a pre-determined exposure threshold, greater than or equal to a predetermined exposure threshold, or a dose than cannot be determined.

The classification method 120 may receive, as input, measured intensities of a plurality of biomarkers (for example, up to six biomarkers (i=1 . . . 6)) and one or more control lines (for example, two control lines (j=1 . . . 2)) distributed across one or more LFA test strips. The one or more LFA test strips may be contained within an LFA cassette. The LFA cassette may be configured with an RFID tag. The classification method 120 may utilize a standard curve that converts measured intensities to biomarker concentrations. The standard curve may be empirically determined during manufacture of the LFA test strips by comparing reference data on biomarker concentration to intensities measured by an analyzer. Since the standard curve may vary across different lots of LFA test strips, the applicable standard curve may be encoded on the RFID tag located on the LFA cassette. The standard curve may be stored as a parametric function on the RFID tag. Biomarker concentrations output by the standard curve may be used as the inputs to the classification method 120.

The classification method 120 may receive the biomarker concentrations and determine (estimate) a Test Statistic for a human subject. For a blood sample j, the Test Statistic may be determined as the sum across biomarkers of −ln(Pᵢⱼ*) where: 1) i denotes biomarker, and 2) the Pᵢⱼ* are estimates obtained by determining a probability Pᵢⱼ that a person from a reference population (e.g., humans age 12 or above who had not been exposed to radiation) would have a biomarker value (in ng/ml) above the value obtained from the j-th blood sample. A Test Statistic threshold may be determined by determining Test Statistics for samples from the reference population and the distribution may be determined. The Test Statistic threshold may be obtained such that no more than 5% of Test Statistic values from the reference population are above the Test Statistic threshold. In an embodiment, the Test Statistic threshold may be determined by a computing device and the Test Statistic threshold loaded onto the RFID tag. The Test Statistic estimated from the received biomarker concentrations may then be determined and compared to the Test Statistic threshold. The classification method 120 then uses the estimated test statistic (TS) and the Test Statistic threshold to reach one of two results 130: TS<=Test Statistic threshold is a Negative result (consistent with no radiation exposure); TS>Test Statistic threshold is a Positive result (consistent with radiation exposure sufficient to distinguish the observation from an unexposed population and having a moderate to high probability of occurring if the exposure is 2 Gy or greater).

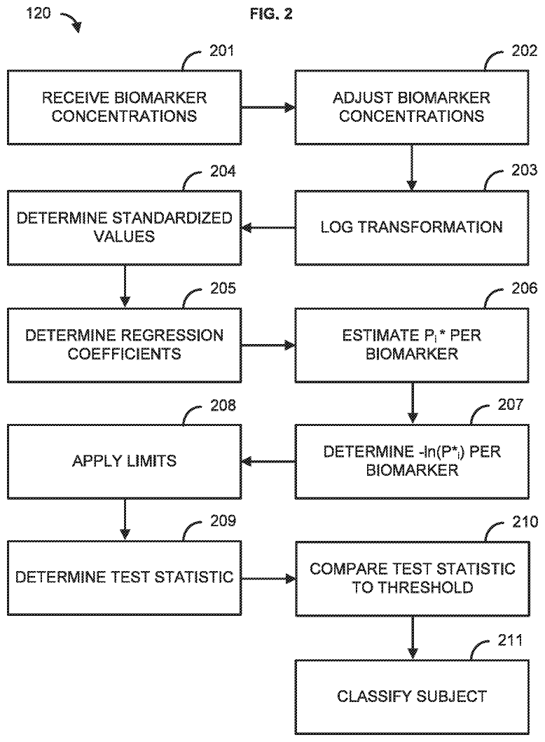

FIG. 2 shows an example classification method 120. At 201, one or more biomarker concentration values may be received. The biomarker concentration values may be received from the concentration method 110. Let Cᵢ denote the concentration (in ng/ml) of the i-th biomarker. Because the classification method 120 may be derived using a data set of ELISA measurements from frozen venous plasma samples, the biomarker concentration values obtained by fingerstick LFA or SYS measurement on whole blood that are input into the classification method 120 may be adjusted to approximate the type of measurements used in deriving the classification method 120.

At 202, biomarker concentration values may be adjusted. In an embodiment, any biomarker concentration values that are less than the Limit of Detection (LoD) may be set to be equal to the LoD detection amount. Examples of the LoD detection amount include concentrations in the range from 10 pg/ml to 10 ng/ml, and may depend on the sensitivity of the assay. LoD refers to the smallest concentration value that can be reliably measured by an analytical procedure. In another embodiment, the classification method 120 may adjust concentrations Cᵢ that are less than a biomarker-specific minimum value (C_{i-Min}) to equal that minimum value. The value of C_{i-Min} is a physiological minimum value of any particular protein, and will vary from protein to protein. The minimum value for the i-th biomarker may be stored on a data source such as the RFID tag as biomarker_min_Ci_value. Table 1 below provides example parameters that may be stored on the RFID tag. In an embodiment, C_{i-Min} may be set to the 5^{th} percentile of a distribution from a reference population. For example, for AMY1A the 5^{th} percentile for NHP is 309 ng/ml and for humans is 19.81 ng/ml. Step 202 may safeguard against the presence of negative concentration values. In the event that there is a requirement that values less than the LoD be set equal to the LoD, the biomarker_min_Ci_values could be set equal to the LoD. Note that if Cᵢ is at or below its minimum value for a biomarker, steps 203-207 may optionally be skipped for that biomarker.

At 203, a natural log transform may be performed on the biomarker concentration values, resulting in a value Lᵢ. Lᵢ denotes ln(Cᵢ). This creates an approximation to a “normal” distribution for subsequent statistical operations.

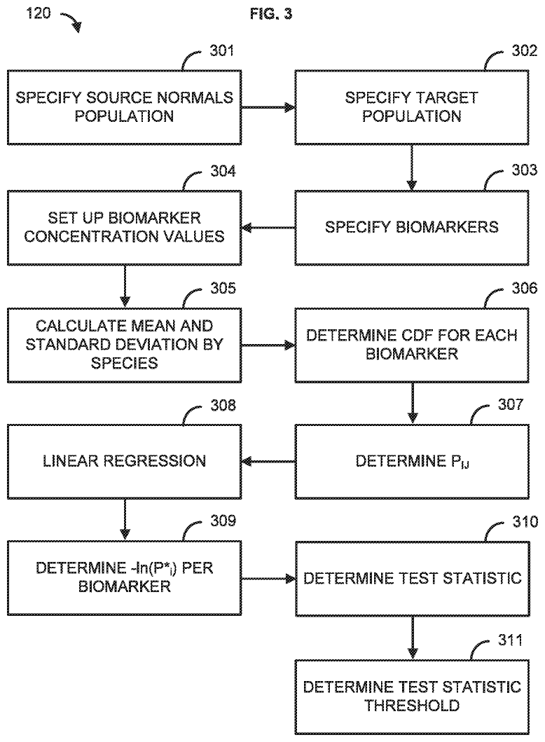

At 204, standardized biomarker concentration values may be determined by evaluating Zᵢ=(Lᵢ−Mᵢ)/Sᵢ, wherein i indexes the biomarkers. The mean (Mᵢ) and standard deviation (Sᵢ) values for each natural log transformed biomarker may be previously determined (e.g., from human “normals” or baseline NHP observations), and the human values may be stored on a data source, such as the RFID tag of the cartridge. For example, the mean Mᵢ may be stored on the RFID tag as “biomarker_normal_mean” and the standard deviation Sᵢ may be stored as “biomarker_normal_std”. FIG. 3 below provides further details related to determination of Mᵢ and Sᵢ values.

At 205, linear regression coefficients may be determined. The linear regression coefficients may be previously determined and stored on a data source such as the RFID tag. The linear regression coefficients may be retrieved from the RFID tag. Linear regression coefficients may comprise Aᵢ and Bᵢ in order to calculate Pᵢ as one minus a cumulative distribution function (CDF) value of the i-th biomarker. FIG. 3 below provides further details related to determination of the linear regression coefficients. For example, the coefficients Aᵢ and Bᵢ may be stored on the RFID tag as “biomarker_coefficient_a” and “biomarker_coefficient_b”.

At 206, Pᵢ may be estimated as Pᵢ*=exp(Aᵢ+BᵢZᵢ)/(1+exp(Aᵢ+BᵢZᵢ)). Estimation of Pᵢ provides a technical improvement that addresses limited memory capacity issues associated with a biodosimeter/analyzer that limit the ability to store the entire distribution of Pᵢ for a reference population.

At 207, the negative of the natural logarithm of the upper tail probability for each biomarker may be determined as −ln(Pᵢ*). The star indicates that Pᵢ* is derived from the regression equation rather than being directly from the empirical Pᵢ.

At 208, biomarker-specific limits, if any, for values of −ln(Pᵢ*) may be read from the RFID tag and applied. Both upper and lower limits may be applied. Upper limits would be applied if it was desired that no single biomarker contribute more to the test statistic than that limit. In an embodiment, no biomarker specific limit is required. For example, if selected biomarkers have high values on different days. If a group of biomarkers reached their peaks on the same day, it might be worthwhile capping the contribution of each individually so that more than a single biomarker would need to be elevated to trigger a positive classification. For example, if the test statistic threshold was 8, and it was desired to cap the contribution of two of three biomarkers, the contribution of one biomarker (e.g., that biomarker's value of −ln(Pi*)) could be capped at 6, another biomarker's value at 5, and a third biomarker's value not capped at all. Whether or not this would be desirable would need to be determined for the specific set of biomarkers and application of the algorithm. Lower limits could also be imposed. For example, if −ln(Pᵢ*) <LoD the value of −ln(Pᵢ*) may be set a predetermined value. In an embodiment, a lower limit of the 5^{th} percentile of the distribution of the biomarker in an unexposed population may be applied. If a biomarker has a value less than the 5^{th} percentile, the value of −ln(Pi*) is set to −ln(1−0.05)=0.051. In an embodiment, for Cᵢ values less than a biomarker minimum concentration value (e.g., stored as biomarker_min_Ci_value on the RFID tag), −ln(Pᵢ*) may be set to a minimum value (ln(Pᵢ*)_{Min}) for the negative of the natural logarithm of the upper tail probability for each biomarker (e.g., stored on the RFID tag as biomarker_min_LnPi_value). For example, if a biomarker minimum concentration value is the 5^{th} percentile of the reference population, the minimum value (ln(Pᵢ*)min) will be set to −ln(0.975) (e.g., the value for −ln(Pᵢ) that would be obtained if the Ci were at the 2.5^{th} percentile of the reference population).

In an embodiment, if a maximum value (ln(Pᵢ*)_{Max}) for the negative of the natural logarithm of the upper tail probability for each biomarker (e.g., stored on the RFID tag as biomarker_max_LnPi_value) is greater than 0, and Cᵢ is above the biomarker minimum concentration value (biomarker_min_Ci_value), then −ln(Pᵢ*) may be set to ln(Pᵢ*)_{Max}. In most instances, ln(Pᵢ*)_{Max} is expected to be set equal to −99, in which case this adjustment can be ignored. However, if the biomarker minimum concentration value is set too high (for example, which may occur when a minimum value for a biomarker concentrations is set to the LoD value), then there may be too few observations available to calculate Aᵢ and Bᵢ regression coefficients (which will be set to zero). In this case ln(Pᵢ*)_{Max} will not be set equal to −99 and all Cᵢ values above the biomarker minimum concentration value will have their corresponding −ln(Pᵢ*) values set to ln(Pᵢ*)_{Max}.

In an embodiment, if ln(Pᵢ*)_{Max} value is greater than 0 and Cᵢ is above the biomarker minimum concentration value for a specific biomarker, then for this biomarker steps 203 to 208 may be optionally skipped.

In an embodiment, if −ln(Pᵢ*) is greater than a threshold value −ln(Pᵢ*)_{Th} for a biomarker (e.g., stored on the RFID tag as biomarker_LnPi_threshold), then −ln(Pᵢ*) may be set to −ln(Pᵢ*)_{Th}. In most instances, −ln(Pᵢ*)_{Th} will be set to 999, which effectively means that this parameter is unlikely to affect the outcome of the classification method 120 (e.g., because the Test Statistic threshold value described below may have a value less than 10). The parameter −ln(Pᵢ*)_{Th} is available if it is desired that no single biomarker should be sufficiently large to result in the Test Statistic for a subject to exceed the Test Statistic threshold value.

At 209, a Test Statistic (TS) may be determined for the human subject. The Test Statistic may be determined by summing the −ln(Pj*) values across the biomarkers.

At 210, the determined TS for the human subject may be compared with a

Test Statistic Threshold value X (e.g., which may be stored on the RFID tag as test_statistic_threshold). The Test Statistic Threshold may be previously determined, stored on the RFID tag, and retrieved for the comparison. Further details related to determination of the Test Statistic Threshold value are provided below. The Test Statistic Threshold value may be determined such that Y % of normal humans will have a Test Statistic less than X (for example, Y=95%). The false positive rate for normal humans would thus be 1−Y (e.g., 5%).

At 211, the determined Test Statistic for the human subject may then be classified based on the comparison to the Test Statistic Threshold value. A subject may be classified as negative for the condition if determined Test Statistic is less than X and may be classified as positive for the condition if determined Test Statistic is greater than or equal to X. A subject may be classified as negative for the condition if determined Test Statistic is less than or equal to X and may be classified as positive for the condition if determined Test Statistic is greater than X. A result of the classification, Positive or Negative, may be output to a display of the biodosimeter/analyzer. In an embodiment where the condition comprises exposure to certain levels of radiation, an observation as positive indicates that the human subject has been exposed to 2 or more Gy an observation as negative indicates that the human subject has not been exposed or has been exposed to less than 2 Gy.

FIG. 3 shows a method 300 for determining parameters used in the classification method 120. At 301, normals for a source population may be determined. The term “source normal” may refer to all observations (the set of biomarker concentrations at a specific time, for a specific individual) that will be used in the source population. When NHP are the source population, the normals may comprise all baseline observations before radiation exposure. When humans are the source population, the normals may be humans age 12 and above from the general population (e.g., not selected specifically for a medical condition or because of unusual radiation exposure).

At 302, the target population may be determined. This is the population to which the algorithm will be applied and the observations classified as “positive” or “negative”. In our research “positive” for humans means 2 or more Gy of radiation exposure and for NHP means 4 or more Gy of radiation. A “normals” subset of the target population may be used in a subsequent step to standardize the target population (and should be used if the source and target populations are different species).

At 303, the biomarkers to be used may be specified, along with relative weights for the biomarkers. These relative weights allow some biomarkers to have increased or decreased importance. These weights may be denoted as “biomarker weights.” By way of example, the biomarkers specified may comprise AMY1, FLT3L, and MCP1 and the biomarkers weights are all unity (e.g., all biomarkers have the same weight, which is set to 1.0).

At 304 the biomarker concentration values may be set up. For example, it may be determined whether each biomarker concentration value is larger or smaller in the observations that are positive relative to those that are negative. Without loss of generality it may be assumed that biomarker concentration values for the specified biomarkers increase with exposure to radiation. If biomarker concentration values of a specified biomarker instead decrease with exposure to radiation, then these biomarker concentration values may be replaced by the inverse (or a different transformation that accomplishes this purpose). The resulting biomarker concentration values now increase with exposure to radiation. Biomarkers should behave the same in the source and target populations (e.g., increase in both or decrease in both populations with radiation exposure).

By way of further example, it may be determined if extrapolation weights should be used to extrapolate the “source” observations on normals to a larger population. When the source population is human, gender and age-specific weights may be used extrapolate results to the US population age 12 and over. If there is no larger population to extrapolate to, then the extrapolation weights can all be equal. In subsequent steps, the term “weighted” shall mean using either equal or unequal extrapolation weights. Extrapolation weights may be standardized to sum to 1.0 across the source normals.

Optionally, the biomarker concentration values may be log transformed.

At 305, if the source and target populations are different species (for example, if the source are NHP and the target are humans) the observations may be standardized. Standardization is optional if the source and target populations are the same species. Then for each of the two populations, determine the weighted mean (Mᵢ) and weighted standard deviation (Sᵢ) of the (possibly transformed) biomarker concentration values in “normal.” The calculated mean (Mᵢ) for each population and the calculated standard deviation (Sᵢ) for each population may be stored.

If performing standardization, then subtract the calculated mean from each observation (normals and non-normals) and divide by the calculated standard deviation. For each population, the standardized values may be evaluated by, Zᵢⱼ=(Cᵢⱼ−Mᵢⱼ)/Sᵢⱼ), where i indexes observations and j indexes biomarkers.

At 306, for each biomarker, the weighted cumulative distribution functions (CDF) for the standardized source observations may be determined. If the biomarker concentration values are sorted from smallest to largest, and the weights are normalized to sum to 1.0, then the CDF at the m-th observation may be defined as the sum of the weights from 1 to m. This value may be multiplied by (N/(N+1)) where N is the total number of observations, to reduce issues that may otherwise occur in later steps with a log transformation.

At 307, Pᵢⱼ may be determined as one minus the CDF value at the i-th observation of the j-th biomarker. For example, if there are 99 observations in the source distribution, and they are equally weighted, then for the j-th biomarker, the observation with: the largest value for that biomarker (e.g., the 1st value if the observations are ordered from largest to smallest) will have a Pᵢⱼ value of 1/100; the second largest value will have a Pᵢⱼ value equal to 2/100; and the smallest value will have a Pᵢⱼ value of 99/100.

At 308, for the j-th biomarker, conduct a weighed linear regression where the dependent variable is the logit of the upper tail probability (e.g., ln((Pᵢⱼ)/(1−Pᵢⱼ)) and the independent variables are a constant and the standardized biomarker values Zᵢⱼ. The observations in this regression are from the source normals. This yields regression coefficients “Aⱼ” for the constant and “Bⱼ” for the coefficient of the j-th standardized biomarker value. Pᵢⱼ may be estimated as Pᵢⱼ*=(exp(Aⱼ+BⱼZᵢⱼ)/(1+exp(Aⱼ+BⱼZᵢⱼ).

In an embodiment, given biomarker i, Aᵢ and Bᵢ are the coefficients in a weighted linear regression where the independent variables are a constant and Zᵢ, the dependent variable is ln((Pᵢ)/(1−Pᵢ)), and Pᵢ is the empirical probability that an observation of the i-th biomarker from the reference population is greater than Zᵢ. This yields regression coefficients Aⱼ for the constant and Bⱼ for the coefficient of the i-th standardized biomarker concentration value, which may be stored as a look up table in the RFID tag.

At 309, the negative of the natural logarithm of the upper tail probability for each biomarker may be determined as −ln(Pᵢⱼ*) where the star indicates that the Pᵢⱼ* are derived from the regression equation rather than being directly from the empirical Pᵢⱼ.

Optionally, limits may be applied, such as a biomarker-specific maximum for −ln(Pᵢⱼ*). For example a maximum value for the j-th biomarker may be specified as 4.0 in which case the estimated value for −ln(Pᵢⱼ*) for that biomarker would be replaced by min(4.0, −ln(Pᵢⱼ*)). Biomarker-specific maximums can be useful for restraining the effect of a particular biomarker (so a large value of one biomarker might not be sufficient to declare an observation as “positive’).

At 310, test statistics may be determined. Test statistics may be determined for each observation (including those in the target population) by summing the −ln(Pᵢⱼ*) values across the biomarkers. Note that the −ln(Pᵢⱼ*) values for the target population are derived from the regression coefficients obtained using source normals.

At 311, a Test Statistic Threshold value may be determined. The Test Statistic Threshold value (X) may be determined by determining a value X such that the sum of the weights of source observations where TS is greater than X is equal to the desired false positive rate (FPR) for the source population. In the absence of an indeterminate zone, an observation may be classified as negative if TS is less than or equal to X and is classified as positive if TS is greater than X. If there is an indeterminate zone, the amount of probability in that zone may be specified. For example, if the indeterminate zone is to contain 3% of the source normals, and the FPR is nominally set to 5%, then a value X_upper may be determined such 3.5% of source normals are above X_upper (i.e., 5%−3%/2) and a value X_lower may be determined such that 6.5% of source normals are above X_lower (i.e., 5%+3%0.2). Values between X_lower and X_upper may be classified as “indeterminate.” Values above X_upper may be classified as positive and values below X_lower may be classified as negative.

The Test Statistic Threshold value is determined such that alpha percent (e.g., 5%) of the reference population of humans will have a TS greater than the threshold. The false positive rate for the reference population samples is thus alpha. The false positive rate for radiation exposures greater than 0 and less than 2 Gy will be greater than alpha.

The parameters thus determined by the method 300 may be stored in a data store, such as the RFID tag, of the biodosimeter/analyzer, and utilized by the classification method 120.

In an embodiment, a rapid diagnostic test (RDT) apparatus is disclosed for measuring intensity of optical signals from test and control lines on one or more LFA test strips and determining whether an individual has a condition (e.g., exposure to a radiation dose a) less than a pre-determined exposure threshold (Negative Result), or b) greater than or equal to a predetermined exposure threshold (Positive Result)). An RDT is a medical diagnostic test that is quick and easy to perform. RDTs are suitable for preliminary or emergency medical screening, for use in medical facilities with limited resources, and offer a useful alternative to microscopy in situations where reliable diagnosis using these other analyses tools is not available or where there is a dirth of trained personnel. They also allow point-of-care (POC) testing in primary care in situations where formerly only a laboratory test could provide a diagnosis. RDTs do not require clinical diagnostic methods, such as enzyme-linked immunosorbent assay (ELISA) or polymerase chain reaction (PCR), can be performed independent of laboratory equipment by minimally trained personnel, and deliver results quickly.

The described RDT employs a dipstick or cassette format. A biological specimen (such as a blood) collected from a patient is applied to a sample pad on the test strip (or card) along with certain reagents. After a length of time (depending on the test), the presence of specific bands in the test strip (card) window indicates whether a certain antigen of interest is present in the patient's sample. Typically, a drop of sample (e.g., blood) is added to the RDT through one hole (sample well), and then a number of drops of buffer are usually added through another hole (buffer well). The buffer carries the sample along the length of the RDT. Lateral flow assays are an important tool in RDT.

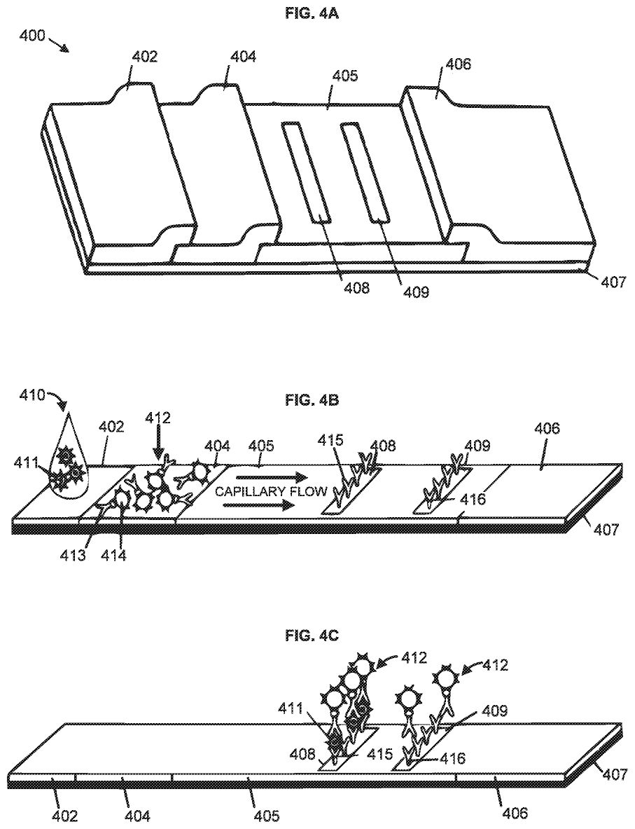

Lateral flow assay test kits are currently available for testing for a wide variety of medical and environmental conditions or compounds, such as a hormone, a metabolite, a toxin, a pathogen-derived antigen, or other biomarkers. FIG. 4A shows a lateral flow test strip 400 that includes a sample receiving zone 402, a labeling zone 404, a detection zone 405, and an absorbent zone 406 on a common substrate 407. These zones 402-406 typically are made of a material (e.g., chemically-treated nitrocellulose) that allows fluid to flow from the sample receiving zone 402 to the absorbent zone 406 by capillary action. The detection zone 405 includes a test region 408 for detecting the presence of a target biomarker in a fluid sample and a control region 409 for indicating the completion of an assay test.

FIG. 4B and FIG. 4C show an assay performed by an example implementation of the test strip 400. A fluid sample 410 (e.g., blood, urine, or saliva) is applied to the sample receiving zone 402. In the example shown in FIG. 4B and FIG. 4C, the fluid sample 410 includes a target biomarker 411 (e.g., a molecule or compound that can be assayed by the test strip 400). Capillary action draws the liquid sample 410 downstream into the labeling zone 404, which contains a substance 412 for indirect labeling of the target biomarker 411. In the illustrated example, the labeling substance 412 comprises an immunoglobulin 413 with an attached dye molecule 414. The immunoglobulin 413 specifically binds the target biomarker 411 to form a labeled target biomarker complex. In some other implementations, the labeling substance 412 is a non-immunoglobulin labeled compound that specifically binds the target biomarker 411 to form a labeled target biomarker complex.

The labeled target biomarker complexes, along with excess quantities of the labeling substance 412, are carried along the lateral flow path into the test region 408 of the detection zone 405, which contains immobilized compounds 415 that are capable of specifically binding the target biomarker 411. In the illustrated example, the immobilized compounds 415 are immunoglobulins that specifically bind the labeled target biomarker complexes and thereby retain the labeled target biomarker complexes in the test region 408. The presence of the labeled biomarker in the sample typically is evidenced by a visually detectable coloring of the test region 408 that appears as a result of the accumulation of the labeling substance in the test region 408.

The control region 409 is designed to indicate that an assay has been performed to completion. Compounds 416 in the control region 409 bind and retain the labeling substance 412. The labeling substance 412 typically becomes visible in the control region 409 after a sufficient quantity of the labeling substance 412 has accumulated. When the target biomarker 411 is not present in the sample, the test region 408 will not be colored, whereas the control region 409 will be colored to indicate that assay has been performed. The absorbent zone 406 captures excess quantities of the fluid sample 410.

Optical inspection of the test region 408 and/or the control region 409 can be used to provide quantitative assay measurements of biomarker concentrations.

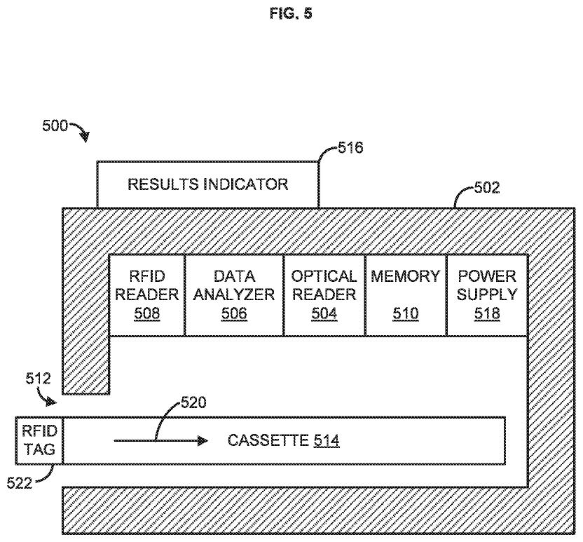

FIG. 5 shows an embodiment of a diagnostic test system 500 that includes a housing 502, an optical reader 504, a data analyzer 506, an RFID reader 508, and a memory 510. A power supply 518 supplies power to the active components of the diagnostic test system 500, including the optical reader 504, the data analyzer 506, the RFID reader 508, and the results indicator 516. The power supply 518 may be implemented by, for example, a replaceable battery or a rechargeable battery.

The housing 502 includes a port 512 for receiving an LFA test strip cassette 514. The LFA test strip cassette 514 may comprise one or more LFA test strips. The LFA test strip cassette 514 may comprise an RFID tag 522 (or other data source accessible to the diagnostic test system 500). When the test strip cassette 514 is loaded in the port 512, the optical reader 504 obtains light intensity measurements from the test strip cassette 514. In general, the light intensity measurements may be unfiltered or they may be filtered in terms of at least one of wavelength and polarization. The data analyzer 506 may perform one or more methods as described herein. In an embodiment, the data analyzer 506 may perform the concentration method 110 on the light intensity measurements and the classification method 120 on the output of the concentration method 110. A results indicator 516 provides an indication of one or more of the results of the method(s) performed by the data analyzer 506. In some implementations, the diagnostic test system 500 is fabricated from relatively inexpensive components enabling it to be used for disposable or single-use applications.

The housing 502 may be made of any one of a wide variety of materials, including plastic and metal. The housing 502 forms a protective enclosure for the optical reader 504, the data analyzer 506, the power supply 518, and other components of the diagnostic test system 500. The housing 502 also defines a receptacle that mechanically registers the test strip cassette 514 with respect to the optical reader 504. The receptacle may be designed to receive any one of a wide variety of different types of test strips and test strip cassettes 514, including test strips of the type shown in FIG. 4A.

In general, each of the test strip cassettes 514 supports lateral flow of a fluid sample along a lateral flow direction 520 and includes a labeling zone containing a labeling substance that binds a label to a target biomarker and a detection zone that includes at least one test region containing an immobilized substance that binds the target biomarker. One or more areas of the detection zone, including at least a portion of the test region and/or a control region, are exposed for optical inspection by the optical reader 504. The exposed areas of the detection zone may or may not be covered by an optically transparent window.

The optical reader 504 includes one or more optoelectronic components for optically inspecting the exposed areas of the detection zone of the test strip cassette 514. In some implementations, the optical reader 504 includes at least one light source and at least one light detector. In some implementations, the light source may include a semiconductor light-emitting diode and the light detector may include a semiconductor photodiode. Depending on the nature of the label that is used by the test strip cassette 514, the light source may be designed to emit light within a particular wavelength range or light with a particular polarization. For example, if the label is a fluorescent label, such as a quantum dot, the light source would be designed to illuminate the exposed areas of the detection zone of the test strip cassette 514 with light in a wavelength range that induces fluorescence from the label. Similarly, the light detector may be designed to selectively capture light from the exposed areas of the detection zone. For example, if the label is a fluorescent label, the light detector would be designed to selectively capture light within the wavelength range of the fluorescent light emitted by the label or with light of a particular polarization. On the other hand, if the label is a reflective-type label, the light detector would be designed to selectively capture light within the wavelength range of the light emitted by the light source. To these ends, the light detector may include one or more optical filters that define the wavelength ranges or polarizations axes of the captured light.

In another approach, the optical reader 504 may generate a baseline of signal intensity from the measurement zones by interpolating between values of the detection signal outside of the measurement zones (e.g., control) and inside of the detection zone. The value of signal intensity representative of the immobilized labeled target biomarker complex may be quantified with respect to the baseline.

The data analyzer 506 processes the light intensity measurements that are obtained by the optical reader 504. In general, the data analyzer 506 may be implemented in any computing or processing environment, including in digital electronic circuitry or in computer hardware, firmware, or software. In some embodiments, the data analyzer 506 includes a processor (e.g., a microcontroller, a microprocessor, or ASIC) and an analog-to-digital converter. In the illustrated embodiment, the data analyzer 506 is incorporated within the housing 502 of the diagnostic test system 500. In other embodiments, the data analyzer 506 is located in a separate device, such as a computer, that may communicate with the diagnostic test system 500 over a wired or wireless connection.

The data analyzer 506 may engage the RFID reader 508 to obtain data from the RFID tag 522 on the test strip cassette 514. The RFID reader 508 may be configured to transmits information, via a wireless air interface, to one or more RFID tags 522. The air interface enables the RFID reader 508 to provide power, query data, and timing information to the RFID tag 522, responsive to which the RFID tag 522 may provide response data. Specifically, the RFID tag 522 may scavenge power from a received radio-frequency (RF) signal and may backscatter the response data to the RFID reader 508 by modulating the impedance of an associated antenna. In a half-duplex communications embodiment, during a reader-to-tag transmission, the RFID reader 508 may modulate an RF waveform with information (e.g., bits). During a tag-to-reader transmission, the RFID reader 508 transmits a Continuous-Wave (CW) radio-frequency signal. The RFID tag 522 then backscatter-modulates the CW signal with bits, creating a radio-frequency (RF) information waveform that is transmitted back to the RFID reader 508.

The RFID tag 522 may be a combination of an RFID circuit (e.g., an RFID Integrated Circuit (IC)), and a coupled antenna (or antennae) to facilitate the reception and transmission of radio-frequency signals via the air interface. The RFID circuit and the antenna are typically located on a base material or substrate (e.g., a plastic or paper material) to thereby constitute the RFID tag 522. The RFID tag 522 may include a number of subcomponents, any one or more of which may be implemented on one or more integrated circuits that form part of the RFID tag 522. Specifically, the RFID tag 522 may include components to facilitate the processing of radio-frequency signals received via the coupled antenna, and also to facilitate the transmission of a radio-frequency signal (e.g., a modulated backscatter signal) via the coupled antenna. A core operates to control operations and states of the RFID tag 522, while a memory stores, inter alia, one or more of, a tag identifier, a product identifier, configuration values applicable to configuration of the RFID tag 522, parameters for performing one or more methods disclosed herein, one or more algorithms, and the like. The RFID tag 522 may be a “passive” tag that scavenges power from a radio-signal received via the air interface. Alternatively, the RFID tag 522 may be an “active” tag and include a power source to power the RFID tag 522.

The RFID tag 522 may be configured to store parameters that are used to calculate a Test Statistic (TS) for a human subject and compare the TS to a Test Statistic threshold value. Table 1 below provides example parameters that may be stored on the RFID tag 522.

TABLE 1
RFID Tag Parameters

| | Number | |
|---|---|---|
| | of | |
| Parameter | Values | Description |
| biomarker_min_Cij_value | 3 | A minimum value for the |
| | | i-th biomarker concentration |
| | | Cᵢ. Measured concentrations |
| | | below this value are set |
| | | equal to this value. |
| biomarker_normal_mean | 3 | The mean value of the |
| | | natural log of biomarker |
| | | concentrations in the |
| | | reference population |
| biomarker_normal_std | 3 | The standard deviation of |
| | | the natural log of biomarker |
| | | concentrations in the |
| | | reference population |
| biomarker_coefficient_a | 3 | A regression coefficient |
| | | for a constant used to |
| | | estimate the probability |
| | | Pᵢ that an observation |
| | | from the reference population |
| | | exceeds the observed |
| | | concentration. |
| biomarker_coefficient_b | 3 | A regression coefficient for |
| | | standardized values of −ln(Cᵢ) |
| | | used to estimate Pᵢ |
| biomarker_min_LnPi_value | 3 | The value to which −ln(Pᵢ*) |
| | | should be set if the |
| | | biomarker is below |
| | | biomarker_min_Ci_value |
| biomarker_max_LnPi_value | 3 | The value to which −ln(Pᵢ*) |
| | | should be set if the |
| | | biomarker is above the |
| | | biomarker_min_Ci value. This |
| | | parameter is ignored if |
| | | it is negative. |
| biomarker_LnPi_threshold | 3 | An upper threshold for −ln(Pᵢ*) |
| test_statistic_threshold | 1 | The threshold to which the test |
| | | statistic is compared |

In general, the results indicator 516 may include any one of a wide variety of different mechanisms for indicating one or more results of an assay test. In some implementations, the results indicator 516 includes one or more lights (e.g., light-emitting diodes) that are activated to indicate, for example, a positive test result and the completion of the assay test (e.g., when sufficient quantity of labeling substance 412 has accumulated in the control region). In other implementations, the results indicator 516 includes an alphanumeric display (e.g., a two or three character light-emitting diode array) for presenting assay test results. In other embodiments, the results indicator 516 may comprise a display screen (e.g., LCD).

The data analyzer 506 may be configured to receive the parameters stored on the RFID tag 522 via the RFID reader 508. The data analyzer 506 may be configured to perform one or more methods described herein. In an embodiment, the data analyzer 506 may be configured to perform the concentration method 110. In an embodiment, the data analyzer 506 may be configured to perform the classification method 120. In an embodiment, the data analyzer 506 may be configured to perform the concentration method 110 and the classification method 120. In an embodiment, the data analyzer 506 may be configured to perform a classification method 600. In an embodiment, the data analyzer 506 may be configured to perform the concentration method 110 and the classification method 600.

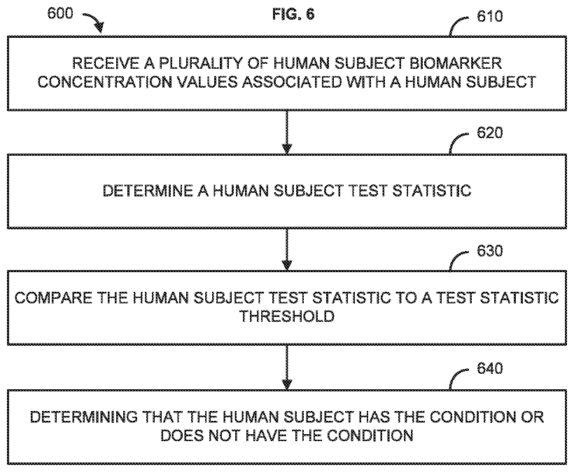

FIG. 6 shows the classification method 600. The classification method 600 may be performed by the data analyzer 506. The classification method 600 may determine a need for a treatment plan for a human subject by assessing a combination of biomarker concentrations previously assessed for a NHP population. In an embodiment, the condition does not possess an easily accessible human study population. The method 600 may comprise receiving (610) a plurality of human subject biomarker concentration values associated with a human subject, wherein the plurality of human subject biomarkers is associated with a condition, determining (620), based on the plurality of human subject biomarker concentration values, a human subject test statistic, comparing (630) the human subject test statistic to a test statistic threshold, wherein the test statistic threshold is derived based in part on non-human primate (NHP) subject data, and determining (640), based on the human subject test statistic exceeding the test statistic threshold, that the human subject has the condition or that the human subject does not have the condition. The test statistic threshold may be derived in part based on NHP subject data and in part based on human subject data.

Receiving the plurality of subject biomarker concentration values may comprise measuring an intensity of light reflected from each of a plurality of zones of a lateral flow assay test strip, wherein each of the plurality of human subject biomarkers is associated with one zone of the plurality of zones, and wherein a control is associated with at least one zone of the plurality of zones and converting, for each zone of the plurality of zones, the intensity of light into a human subject concentration value for the biomarker of the plurality of human subject biomarkers associated with a respective zone of the plurality of zones. The condition may be exposure to radiation at 2Gy or greater.

The plurality of human subject biomarkers may comprise one or more of salivary alpha amylase (AMY1), Flt3 ligand (FLT3L), or monocyte chemotactic protein 1 (MCP1) and wherein the condition is exposure to radiation at 2 Gy or greater. It should be noted that while biomarkers AMY1, FLT3L, and MCP1 have been selected for measuring radiation exposure, the apparatus and associated methods described here can be used for assessing other conditions where a combination of biomarker values are measured and assessed against corresponding threshold values to determine if a subject has or does not have the condition.

Determining, based on the plurality of human subject biomarker concentration values, the human subject test statistic comprises determining a sum across the plurality of human subject biomarker concentration values of −ln(Pᵢⱼ*) wherein i denotes a biomarker, and Pᵢⱼ* is an estimate of a probability (Pᵢⱼ) that a person from a reference population would have a biomarker concentration value above the corresponding human subject biomarker concentration value obtained from a j-th sample of the human subject.

Determining, based on the plurality of human subject biomarker concentration values, the human subject test statistic may comprise for each human subject biomarker concentration value (Cᵢ) of the plurality of human subject biomarker concentration values: determining a natural log transformation (Lᵢ) by evaluating Lᵢ=ln(Cᵢ), determining a standardized value (Zᵢ) by evaluating Zᵢ=(Lᵢ−Mᵢ)/Sᵢ, wherein Mᵢ represents a mean value of a natural log of biomarker concentrations in a reference population and wherein Sᵢ represents a standard deviation of the natural log of biomarker concentrations in the reference population, determining a coefficient Aᵢ and a coefficient Bᵢ, wherein the coefficient Aᵢ comprises a regression coefficient for a constant used to estimate the probability Pᵢ that an observation from a reference population exceeds an observed concentration and wherein the coefficient Bᵢ comprises a regression coefficient for standardized values of −ln(C,) used to estimate Pᵢ, estimating a probability (Pᵢ) as Pᵢ*(Zᵢ, Aᵢ, Bᵢ), determining an inverse natural log transformation of Pᵢ* by evaluating −ln(Pᵢ*), and determining the human subject test statistic by evaluating Σᵢ(−ln(P*ᵢ)).

The method 600 may further comprise determining if Cᵢ is less than a concentration minimum, wherein if Cᵢ is less than the concentration minimum, setting −ln(Pᵢ*) to a first predefined value, determining if Cᵢ is greater than a concentration maximum, wherein if Cᵢ is greater than the concentration maximum, setting −ln(Pᵢ*) to a second predefined value, and determining if −ln(Pᵢ*) is greater than an upper threshold value for acceptable values of −ln(Pᵢ*), wherein if −ln(Pᵢ*) is greater than the upper threshold value, setting −ln(Pᵢ*) to the upper threshold value.

One or more of the test statistic threshold, Mᵢ, Sᵢ, the coefficient Aᵢ, the coefficient Bᵢ, the concentration minimum, the concentration maximum, or the upper threshold value is received via an RFID tag affixed to a cartridge containing a lateral flow assay test strip.

The method 600 may further comprise outputting an indication that the human subject has the condition to a display.

The method 600 may further comprise previously deriving the test statistic threshold based in part on non-human primate (NHP) subject data such that a False Negative Rate is less than 10% for humans exposed to greater than or equal to 3.6 Gy.

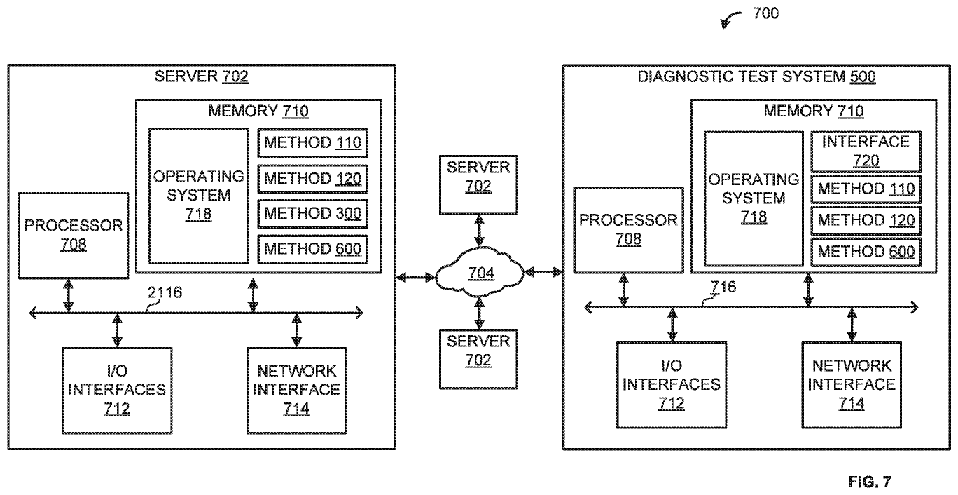

FIG. 7 is a block diagram depicting an environment 700 comprising non-limiting examples of a server 702 and the diagnostic test system 500 connected through a network 704. In an aspect, some or all steps of any described methods may be performed on a computing device as described herein. The server 702 can comprise one or multiple computers configured to store and/or perform one or more of the method 110, the method 120, the method 300, the method 600, parameters generated by the method 110, the method 120, the method 300, the method 600, parameters utilized by the method 110, the method 120, the method 300, the method 600, and the like. The diagnostic test system 500 can comprise one or multiple computers configured to operate a user interface 720 such as, for example, a laptop computer or a desktop computer. Multiple diagnostic test systems 500 can connect to the server(s) 702 through a network 704 such as, for example, the Internet. A user on a diagnostic test system 500 may interact with and/or otherwise cause the method 110, the method 120, and/or the method 600 to execute via with the user interface 720. The user interface 720 may be configured to display a result of the method 110, the method 120, and/or the method 600.

The server 702 and the diagnostic test system 500 can be a digital computer that, in terms of hardware architecture, generally includes a processor 708, memory system 710, input/output (I/O) interfaces 712, and network interfaces 714. These components (708, 710, 712, and 714) are communicatively coupled via a local interface 716. The local interface 716 can be, for example but not limited to, one or more buses or other wired or wireless connections, as is known in the art. The local interface 716 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 708 can be a hardware device for executing software, particularly that stored in memory system 710. The processor 708 can be any custom made or commercially available processor, a central processing unit (CPU), an auxiliary processor among several processors associated with the server 702 and the diagnostic test system 500, a semiconductor-based microprocessor (in the form of a microchip or chip set), or generally any device for executing software instructions. When the server 702 or the diagnostic test system 500 is in operation, the processor 708 can be configured to execute software stored within the memory system 710, to communicate data to and from the memory system 710, and to generally control operations of the server 702 and the diagnostic test system 500 pursuant to the software. The processor 708 of the diagnostic test system 500 may be configured execute software for performing the method 110, the method 120, and/or the method 600. The processor 708 of the server 702 may be configured execute software for performing the method 110, the method 120, the method 300, and/or the method 600.

The I/O interfaces 712 can be used to receive user input from and/or for providing system output to one or more devices or components. User input can be provided via, for example, a keyboard and/or a mouse. System output can be provided via a display device and a printer (not shown). I/O interfaces 712 can include, for example, a serial port, a parallel port, a Small Computer System Interface (SCSI), an IR interface, an RF interface, and/or a universal serial bus (USB) interface. As described previously with regard to FIG. 5, the diagnostic test system 500 may comprise input/output (I/O) interfaces 712 such as an RFID reader and an optical reader.

The network interface 714 can be used to transmit and receive from an external server 702 or a diagnostic test system 500 on a network 704. The network interface 714 may include, for example, a 10 BaseT Ethernet Adaptor, a 100 BaseT Ethernet Adaptor, a LAN PHY Ethernet Adaptor, a Token Ring Adaptor, a wireless network adapter (e.g., WiFi), or any other suitable network interface device. The network interface 714 may include address, control, and/or data connections to enable appropriate communications on the network 704.

The memory system 710 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 710 may incorporate electronic, magnetic, optical, and/or other types of storage media. Note that the memory system 710 can have a distributed architecture, where various components are situated remote from one another, but can be accessed by the processor 708. The memory system 710 may be configured for storing parameters generated by, and/or utilized by, and of the method 110, the method 120, the method 300, and/or the method 600.

The software in memory system 710 may include one or more software programs, each of which comprises an ordered listing of executable instructions for implementing logical functions. In the example of FIG. 7, the software in the memory system 710 of the server 702 can comprise the method 110, the method 120, the method 300, the method 600, and a suitable operating system (O/S) 718. In the example of FIG. 7, the software in the memory system 710 of the diagnostic test system 500 can comprise the method 110, the method 120, the method 600, user interface 720, and a suitable operating system (O/S) 718. The operating system 718 essentially controls the execution of other computer programs, such as the operating system 718, the user interface 720, and provides scheduling, input-output control, file and data management, memory management, and communication control and related services.

For purposes of illustration, application programs and other executable program components such as the operating system 718 are illustrated herein as discrete blocks, although it is recognized that such programs and components can reside at various times in different storage components of the server 702 and/or the diagnostic test system 500. An implementation of the method 110, the method 120, the method 300, the method 600, and/or the user interface 720 can be stored on or transmitted across some form of non-transitory computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

### EXAMPLES

There is a need to rapidly triage individuals for absorbed radiation dose following a significant nuclear event where tens of thousands of individuals will need to be evaluated in a relatively short period of time to ensure effective medical treatment and efficient use of medical resources. Because most exposed individuals will not have physical dosimeters, the described methods may be used to assess exposure dose that is based on the analysis of a specific panel of blood proteins that can be easily obtained from a fingerstick blood sample. A panel of three protein biomarkers has been identified that are upregulated in human patients receiving fractionated doses of total body radiation therapy as a treatment for cancer. These protein biomarkers are salivary alpha amylase (AMY1), Flt3 ligand (FLT3L), and monocyte chemotactic protein 1 (MCP1). Furthermore, these proteins exhibit similar radiation response in non-human primates receiving either single acute or fractionated doses of ionizing radiation. The described methods have been demonstrated the use of this panel of three proteins to classify with high accuracy a data set consisting of 1051 human samples obtained from radiotherapy patients, normal healthy individuals, and several special population groups that include diabetic, obese, arthritic, pregnant, and immune compromised individuals as well as individuals with burns, trauma, and mild infections. The biodosimeter/analyzer described herein can rapidly measure these three proteins in a fingerstick blood sample for use in radiation exposure triage in a mass casualty nuclear event.

The described biodosimeter/analyzer is a point-of-care (POC) radiation biodosimeter that can be used to triage potentially exposed individuals following radiological and nuclear events. The described biodosimeter/analyzer is capable of distinguishing between absorbed doses of <2 Gy and >2 Gy, has high classification accuracy for samples obtained in the 1 to 7-day post exposure time window, performs comparably across the US demographic range for all age groups, and is not be confounded by common medical conditions prevalent in the US population as well as special population groups designated by the Department of Health and Human Services (HHS). Additionally, the device is operable by minimally trained individuals and provide a result in under 30 minutes from a fingerstick blood sample.

A set of host-response plasma were proteins that are indicative of exposure to ionizing radiation at or above a threshold level (which is 2 Gy in humans but different in an animal model). In a companion paper [Balog et al., Development of a point-of-care radiation biodosimeter: studies using novel protein biomarker panels in non-human primates, Int. Jour. Radiation Biology, 2019] the results obtained from three large scale non-human primate (NHP) studies are discussed and a panel of protein biomarkers that are significantly elevated in NHPs in response to acute absorbed doses of ionizing radiation is identified. These biomarkers include alpha-1-antichymotrypsin (AACT), salivary alpha amylase (AMY1), Flt3 ligand (FLT3L), and monocyte chemotactic protein 1 (MCP1). This panel of biomarkers is demonstrated to classify a large data set of NHP blood plasma samples with high accuracy and that the baseline levels of these markers and subsequent levels following significant absorbed doses of radiation can be straightforwardly detected using a lateral flow test. An earlier paper [Bazan et al., Int. J. Radiation Oncology Biol Phys. 90(3):612-9, 2013] discussed some of our initial work on proteins that could be useful radiation biomarkers in humans based on initial observations in human radiotherapy patients. The current paper presents additional results from radiotherapy patients as well as a number of special population groups and compares results with those observed in NHPs and identifies a specific panel of proteins that are useful for radiation biodosimetry.

The three biomarkers AMY1, FLT3L, and MCP1 are significantly up-regulated in human radiotherapy patients receiving fractionated doses of ionizing radiation administered over a period of several days. Using the methods described herein, this panel of 3 biomarkers classified a data set consisting of 1051 human samples with an accuracy of 92%, a sensitivity of 90% and a specificity of 93%. This human data set consists of samples obtained from normal healthy individuals, several special population groups, and human radiotherapy patients who received fractionated doses of total body radiation. The three markers exhibit a radiation response in human radiotherapy patients that is similar to that observed in NHPs.

These three markers have all been previously reported in the literature in the context of radiation injury. Salivary alpha amylase (AMY1) is highly expressed in the salivary gland and is known to be indicative of radiation injury to the parotid gland [Kishima et al., Am J Roentgenol Radium Therapy Nucl Med. 94: 271-291, 1965]. The rise in AMY1 that results from the irradiation of salivary tissue has also been observed to provide a unique biochemical measure of early radiation effect in normal tissue [Guipaud et al., Ann Ist Super Sanita. 45(3): 278-286, 2009]. The post-irradiation increase in AMY1 has been shown to provide a good criterion for triage of accidentally irradiated patients [Citrin et al., Radiation Oncology. 7:64 doi: 10.1186/1748-717X-7-64, 2002] and in radiotherapy patients receiving total body irradiation [Barrett et al., Br. Med. J. 285:170-171, 1982; Junglee et al., Clin Chem 32:609-610, 1986]. Early changes in salivary gland function are similarly marked in patients receiving either accelerated radiotherapy or conventionally fractionated radiation treatment for some head and neck cancers [Guipaud et al., Ann Ist Super Sanita. 45(3): 278-286, 2009; Leslie et al., Radiotherapy and Oncology. 24(1): 27-31, 1992].

Fms-related tyrosine kinase 3 Ligand (FLT3L) is a hematopoietic cytokine that works in synergy with other growth factors to stimulate the proliferation and differentiation of various blood cell progenitors. Plasma FLT3L concentration during the first five days of radiation therapy directly correlates with the radiation dose in a nonhuman primate model [Bertho et al., Intern J Rad Biol. 77:703-712, 2001]. For patients receiving radio- immunotherapy, FLT3L-adjusted red marrow radiation doses correlates with hematologic toxicity. FLT3L is expressed following radiation-induced injury to the bone marrow [Kenins et al., Journal of Experimental Medicine. 205(3):523-531, 2008]. Monitoring of FLT3L correlates with the severity of damage to the main physiological systems in victims exposed accidentally to ionizing radiation [Bertho et al., Radiation Research. 169(5):543-550, 2008; Bertho et al., Biomarkers. 14(2): 94-102, 2009].

Monocyte chemoattractant protein-1 (MCP-1/CCL2) is a potent chemotactic factor for monocytes which is produced by a variety of cell types, either constitutively, or after induction by oxidative stress [Deshmane et al., Journal of Interferon & Cytokine Research. 29(6): 313-326, 2009]. MCP-1 has been demonstrated to recruit monocytes into foci of active inflammation from infectious diseases (e.g., tuberculosis), rheumatologic diseases (e.g., rheumatoid arthritis), and cancers (e.g., breast cancer) [Deshmane et al., Journal of Interferon & Cytokine Research. 29(6): 313-326, 2009]. MCP1 levels have been observed in patients with non-small cell lung cancer (NSCLC) treated with irradiation (60 Gy in 30 fractions over six weeks) [Siva et al., PLOS ONE 9(10): e109560, 2014]. Mean lung radiation dose correlated with a reduction at one hour in plasma levels of MCP-1. Patients who sustained pulmonary toxicity had markedly reduced MCP-1 levels at one hour post radiation treatment when compared to patients without respiratory toxicity. However, MCP-1 concentrations at four weeks were increased in those patients with severe pulmonary toxicity compared to those patients without severe toxicity. Measurement of cytokine concentrations during radiation therapy could help predict lung toxicity.

Ionizing radiation has been shown to induce the expression of MCP-1 in meningioma cell lines [Nalla et al., Cellular Signalling. 23(8): 1299-1310, 2011], rat liver cells [Moriconi et. al., Radiation Research. 169(2):162-169, 2008], and human lung endothelial cells [Gaugler et. al, Radiation Research. 163(5):479-487, 2005].

### Non-Human Primate Studies

NHP samples were obtained from several different irradiation studies. The use of animals and study protocols were approved by the Institutional Animal Care and Use Committee (IACUC) in all participating institutes and by the sponsor. The first large-scale study was conducted at CitoxLab (Montreal, Canada). This was followed by two additional large-scale studies conducted at LBERI (Albuquerque, N. Mex.). The CitoxLab study (SRI M918) consisted of a total of 50 animals (ages ˜4 yrs) assigned to 6 dose groups receiving single acute TBI absorbed doses of 0, 1, 2, 4, 8, and 10 Gy (the 0 Gy sham animals were re-assigned to the 10 Gy group). Each dose group consisted of 10 animals (5M/5F). The animals were exposed to gamma rays from a Co60 source at a dose rate of 60 cGy/min. A total of 300 venous blood samples were collected from all animals at 6 time points: pre-irradiation, post-irradiation (4-12 hours), and on days 1, 2, 3, and 7 post-irradiation. All irradiated groups presented a significant decrease in leucocytes including lymphocyte counts from day 1 to day 7 with dose dependent severity. A decline in neutrophil and platelet counts as well as a decrease in body weights was also observed for the animals exposed to 8 and 10 Gy.

The LBERI studies consisted of two large-scale acute TBI exposure studies similar to the one performed at CitoxLab. These two studies (SRI M073 and M103) consisted of 60 animals (ages ˜4 yrs) assigned to 6 dose groups of 10 animals (5M/5F). In the M073 study the dose groups were 0, 2, 4, 6, 8, and 10 Gy. In the M103 study, the dose groups were 0, 1, 2, 4, 6, and 8 Gy. A total of 294 and 300 venous blood samples were collected in studies M073 and M103 at 5 time points: pre-irradiation, and on days 1, 3, 5, and 7 post-irradiation (the reduced number of samples collected in M073 resulted from an animal in the 10 Gy group being removed from the study). Animals received TBI absorbed doses from a 6 MV LINAC x-ray beam at a dose rate of 50-80 cGy/min.

All irradiated groups presented a decrease in white blood cell counts from day 1 to day 7 with dose dependent severity.

The total NHP acute exposure sample set obtained from all three studies consists of 895 samples from normal (baseline) NHPs as well as NHPs receiving absorbed doses of radiation in the range of 1 to 10 Gy with blood collections in the 1 to 7-day post-irradiation time window.

### Sample Collection

Venous blood was collected using a single BDTM P100 Blood Collection System for preservation of plasma proteins. Tubes that were not collected to the 8 mL volume were identified as a partial collection. Each tube was inverted 8-10 times to thoroughly mix the P100 anticoagulant and then placed inside a ziplock bag on a layer of wet ice inside a Styrofoam container. Each P100 tube containing blood was centrifuged at 1600 g for 30 min. Using a 1000 ill micro pipettor with appropriately sized tips, 500 ill aliquots of plasma were transferred from the top layer in the P100 tubes into the appropriate number of individual screw-cap 1.5-mL microcentrifuge tubes. These aliquot tubes were stored at −80° C. until shipment on dry ice to SRI. All received samples were stored at −80° C. until analysis by mass spectroscopy or immunoassay.

### Sample Analysis Methods

### Tandem Mass Spectrometry for Initial Marker Discovery

LC-MS/MS analysis of samples utilized a label-free, quantitative shotgun (bottom-up) LC-MS/MS proteomics approach [Wang et al., Anal. Chem. 75(18): 4818-26, 2003, Lin et al., Anal. Chem. 78(16):5762-7, 2006]. In this approach, a specific protease enzyme digests a complex mixture of proteins such as whole plasma to produce a mixture of peptides. The peptide mixture is then separated by reversed-phase capillary HPLC connected online to a hybrid Orbitrap mass spectrometer (Thermo Scientific) that has the capability in real chromatographic time to acquire high-resolution, accurate mass measurements of the peptides in full-scan MS mode and obtain sequence information of the peptides in fragmentation MS/MS mode. In this way, thousands of peptides can be profiled and identified simultaneously in a single analysis using automated software packages. While peptide sequence and protein identification are determined through database searching (ByOnic/ComByne, PARC), relative quantitative information is obtained by comparing the corresponding peptide ion current in MS mode from sample to sample (SIEVE, Thermo Scientific). Overall this represents an efficient and unbiased approach to identify candidate biomarkers and was applied extensively here to find proteins whose plasma levels were sensitive to ionizing radiation.

### Immunoassays

Immunoassays were performed in duplicate and utilized either conventional ELISA or the Luminex multiplex platform. Both assay types are performed in a sandwich format (the analyte to be measured is bound between two primary antibodies—the capture antibody and the detection antibody). ELISA assays were performed for 8 different protein targets using commercially available kits. Luminex assays were performed on 35 different proteins using the NHP metabolic and cytokine panels. Each assay plate included one or more plasma sample standards to evaluate assay variability. The CVs ranged from 3.6% to 11.7%, with an average CV of 10%.

### Targeted Quantitative Mass Spectrometry Assays for Marker Verification

For several of the promising-looking proteins that did not have viable commercially available ELISA or Luminex kits, synthetic, stable-isotope labeled peptides were ordered that could be used as standards for quantitative mass spectrometry based on multiple reaction monitoring [Kondrat et al., Anal. Chem. 50:2017-2021, 1978] analysis across a large subset of the samples. This allowed us to include quantitative data for some of these potentially promising proteins in order to investigate their utility in classifying different radiation dose exposure groups.

A summary of the assays performed for each NHP study and the method used for the analysis is provided in Table 2.

TABLE 2

| Study | Proteins | Format |
|---|---|---|
| M918 | ApoC1, CRP, clusterin, elastase, | ELISA |
| | FLT3L, haptoglobin, AMY1, TNC | |
| | amylin (active), C-Peptide, GIP, | Luminex |
| | ghrelin, glucagon, glucagon-like | |
| | peptide-1, insulin, leptin, | |
| | MCP-1, PP, PYY | |
| | TNFa. IL-6, IFNy, IL-18, IL-13, | Luminex |
| | GM-CSF, VEGF, IL-1ra, IL-1b, | |
| | IL-5, IL-12/23(p40), sCD40L, | |
| | IL-15, MIP-1b, MIP-1a, TGFa, | |
| | IL-8, IL-10, MCP-1, IL-17A, | |
| | IL-4, Il-2, G-CSF | |
| | AACT, NGAL | MRM |
| M073 | AMY1, AACT, FLT3L, IL15, | ELISA |
| | NGAL, MCP1, IL18, CRP | |
| M103 | AMY1, AACT, FLT3L, IL15, | ELISA |
| | NGAL, MCP1, IL18, CRP | |

### Statistical Methods

Data analysis was performed using several different analysis packages: the comprehensive statistical analysis package known as R, which is available as freeware and widely used within the biostatistics community, the Matlab Statistics toolpack, and the Stata statistical and data analysis software. Initial data processing consisted of reading in the raw data files produced by the ELISA and Luminex instruments and preparing a master data file consisting of Excel spreadsheets of the data for each protein for each plasma sample. Standard analyses included preparation of boxplots, histograms, assay CVs, correlation tables, and fold-change plots for each protein. Most analyses were performed on log-transformed data as we found the transformed data to be more normally distributed than the untransformed data. Both paired and unpaired t-tests were performed as well as linear regressions to identify proteins that change significantly from baseline as a result of irradiation. Data sets were classified using several supervised classifiers that included logistic regression, support vector machine, and conditional inference trees. Results from some of these analyses are presented in the following sections.

### Results

### Tandem Mass Spectrometry Results

LC-MS/MS analysis of plasma samples from the M918 NHP study identified many new radiation-responsive proteins and confirmed the expected changes of several known radiation-responsive proteins. Radiation responsive proteins observed in this study included haptoglobin, inter-alpha-trypsin inhibitor heavy chain H4, alpha-1-acid glycoprotein 1-like isoform 2, hemopexin, serpin peptidase inhibitor, alpha-1-antichymotrypsin (AACT), C-reactive protein (CRP) and serum amyloid A protein-like isoform 1 (SAA). Among those upregulated proteins, SAA, CRP, haptoglobin and AACT were found to have the largest fold changes by irradiation. Other promising radiation-responsive proteins observed in the MS studies included neutrophil gelatinase-associated lipocalin (NGAL, also known as lipocalin 2), insulin-like growth factor binding protein 4 (IGFBP4), Cystatin-M/Cystatin-6, Iduronate 2-sulfatase isoform 4 (IDS4), Lymphatic vessel endothelial hyaluronic acid receptor 1 (LYVE1), Properdin, and Catalase Isoform 2. Fold changes for these proteins ranged from 2× to greater than 20× at day 7 in the 8 Gy and 10 Gy samples. Table 3 summarizes the proteins we found to be either upregulated or downregulated in plasma based on our LC-MS/MS analysis of M918 NHP samples.

TABLE 3

| | | | | | Maximum |
|---|---|---|---|---|---|
| | | | | | Fold |
| | | | | Up or | Change |
| Protein name | Dose | | Day | Down | Observed |
| serum amyloid A protein | 10 | Gy | 7 | Up | >200* |
| isoform 1 or 2 (SAA1 or | | | | | |
| SAA2) | | | | | |
| c-reactive protein (CRP) | 10 | Gy | 7 | Up | >100* |
| regenerating islet-derived | 8 or 10 | Gy | 7 | Up | >20* |
| 3 alpha (REG3A) | | | | | |
| insulin-like growth factor | 8 or 10 | Gy | 7 | Up | >20* |
| binding protein 4 (IGFBP4) | | | | | |
| alpha-amylase | 8 or 10 | Gy | 1 | Up | >20** |
| basic salivary proline-rich | 8 or 10 | Gy | 1 | Up | >10** |
| protein | | | | | |
| haptoglobin (HP) | 10 | Gy | 7 | Up | >10* |
| alpha-1-antichymotrypsin | 10 | Gy | 7 | Up | 7.1* |
| alpha-1-acid glycoprotein | 10 | Gy | 7 | Up | 5.6* |
| 1 or 2 | | | | | |
| GDH/6PGL endoplasmic | 8 or 10 | Gy | 7 | Up | >5* |
| bifunctional protein (GDH) | | | | | |
| cystatin-B (CSTB) | 8 or 10 | Gy | 7 | Up | >5* |
| lymphatic vessel | 6.5 | Gy | 7 | Up | ~5 |
| endothelial hyaluronic | | | | | |
| acid receptor 1 (LYVE1) | | | | | |
| neutrophil gelatinase- | 8 or 10 | Gy | 7 | Up | ~5* |
| associated lipocalin | | | | | |
| (NGAL, also known as | | | | | |
| lipocalin 2, LCN2) | | | | | |
| lipopolysaccharide-binding | 10 | Gy | 7 | Up | ~3.5* |
| protein (LBP) | | | | | |
| angiotensinogen (AGT) | 10 | Gy | 7 | Up | 3.2* |
| leucine-rich alpha- | 10 | Gy | 7 | Up | 3.2* |
| 2-glycoprotein (LRG1) | | | | | |
| hemopexin-like (HPX) | 10 | Gy | 7 | Up | 2.9* |
| complement component | 10 | Gy | 7 | Up | 2.6* |
| C9 (C9) | | | | | |
| fibrinogen (FGA, | 10 | Gy | 7 | Up | 2.3* |
| FGB, FGG) | | | | | |
| inter-alpha-trypsin | 10 | Gy | 7 | Up | 2.3* |
| inhibitor heavy chain | | | | | |
| H4 (ITIH4) | | | | | |
| inter-alpha-trypsin | 10 | Gy | 7 | Up | 2.1* |
| inhibitor heavy chain | | | | | |
| H3 (ITIH3) | | | | | |
| complement C5 (C5) | 10 | Gy | 7 | Up | 1.8* |
| complement C3, | 10 | Gy | 7 | Up | 1.8* |
| partial (C3) | | | | | |
| complement C4 (C4A, | 10 | Gy | 7 | Up | 1.9* |
| C4B) | | | | | |
| alpha-1-antitrypsin | 10 | Gy | 7 | Up | 1.9* |
| isoform 4 (SERPINA1) | | | | | |
| catalase isoform 2 (CAT) | 6.5 | Gy | 7 | Up | ~2 |
| apolipoprotein A-IV | 10 | Gy | 7 | Down | ~−10* |
| (APOA4) | | | | | |
| galectin-3-binding protein | 6.5 | Gy | 7 | Down | −4.0 |
| isoform 3 (LGALS3BP) | | | | | |
| gelsolin (GSN) | 10 | Gy | 7 | Down | −3.1* |
| iduronate 2-sulfatase | 6.5 | Gy | 7 | Down | ~−2 |
| isoform 4 (IDS) | | | | | |
| properdin-like (also | 6.5 | Gy | 7 | Down | ~−2 |
| known as Complement | | | | | |
| factor P, CFP) | | | | | |

*compared to 1 Gy pool
**compared to pre-irradiation

### Immunoassay Results

### T-Tests and Boxplots

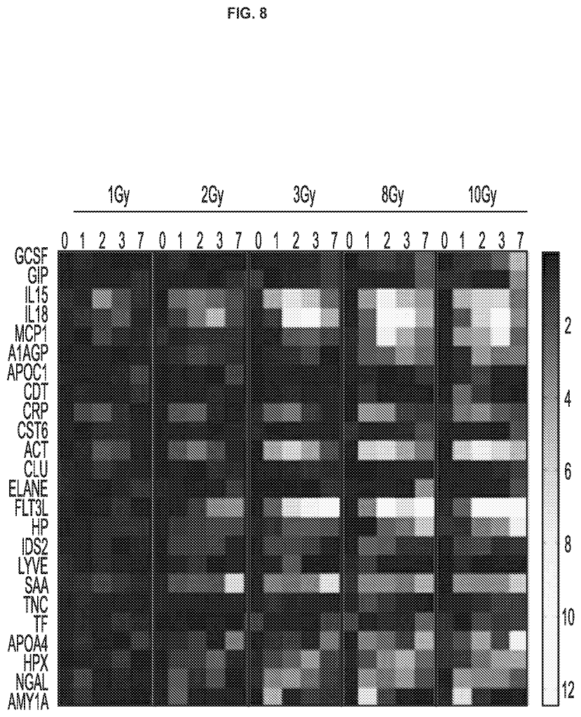

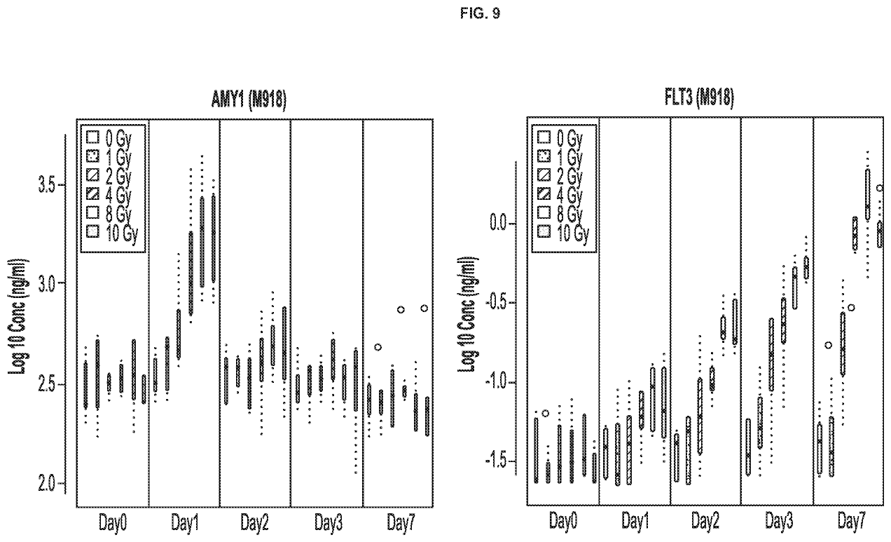

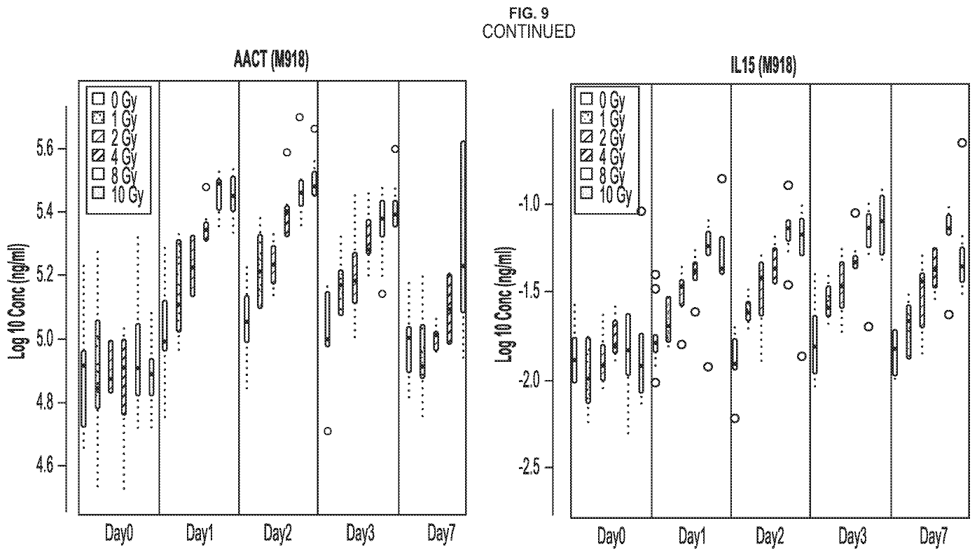

An initial analysis of the M918 immunoassay results consisted of using a t-test to compare the irradiated groups to the control group for each day for each dose group. FIG. 8 shows the results as a heatmap of the log 10 of the t-test p-values for each protein for each day and dose. As can be seen from the heatmap, radiation responsive proteins include IL15, IL18, MCP1, AACT, FLT3L, SAA, NGAL, and AMY1. All showed significant changes following irradiation (p<le-4). FIG. 9 shows boxplots from the M918 immunoassay data for the proteins AMY1A, FLT3L, AACT, and IL15. As can be seen from the plots, all are significantly upregulated from their pre-irradiation values, and each follows a different time course following irradiation. Similar analyses were performed for the M073 and M103 studies confirming the results obtained in the M918 study.

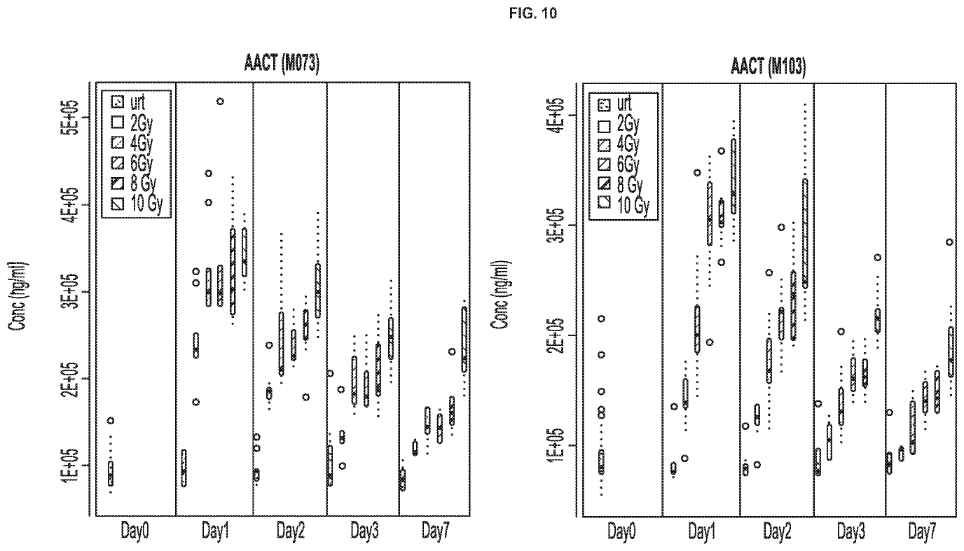

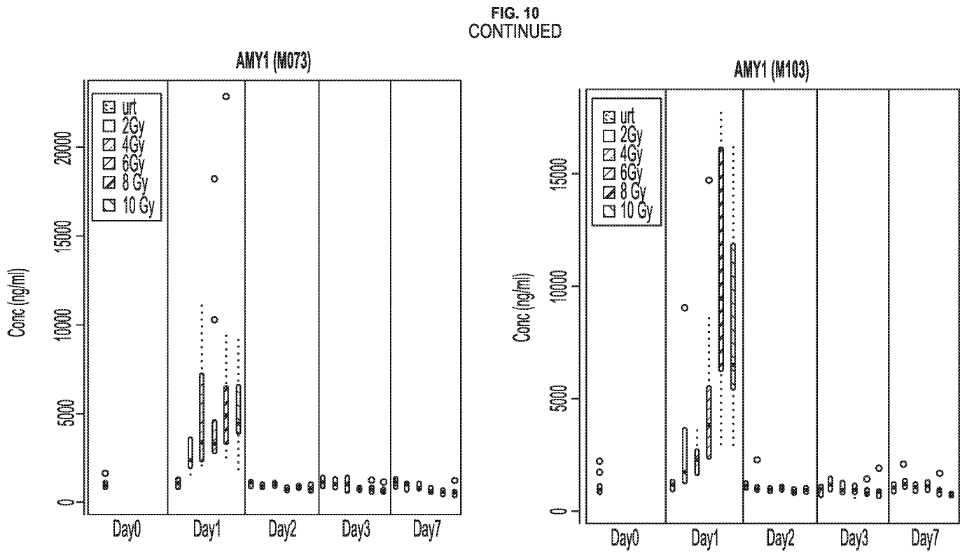

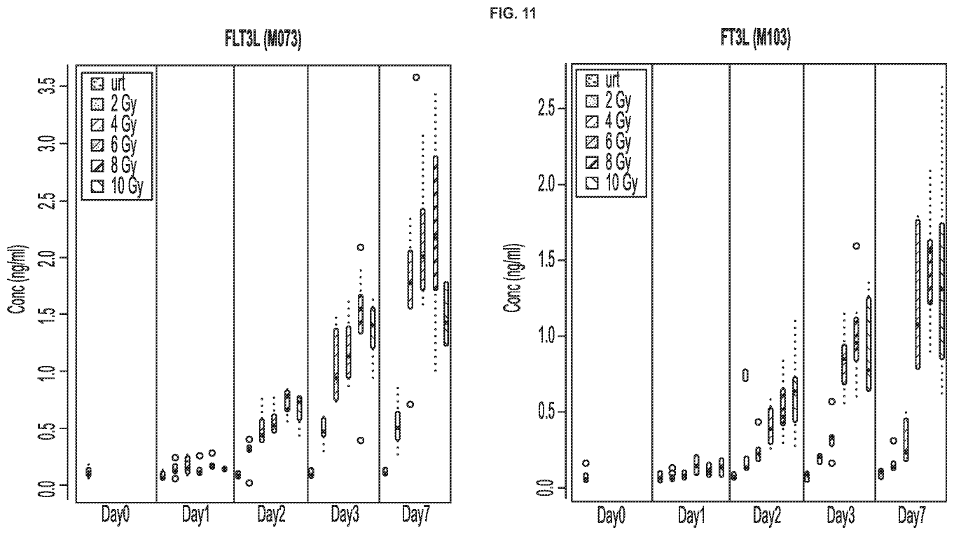

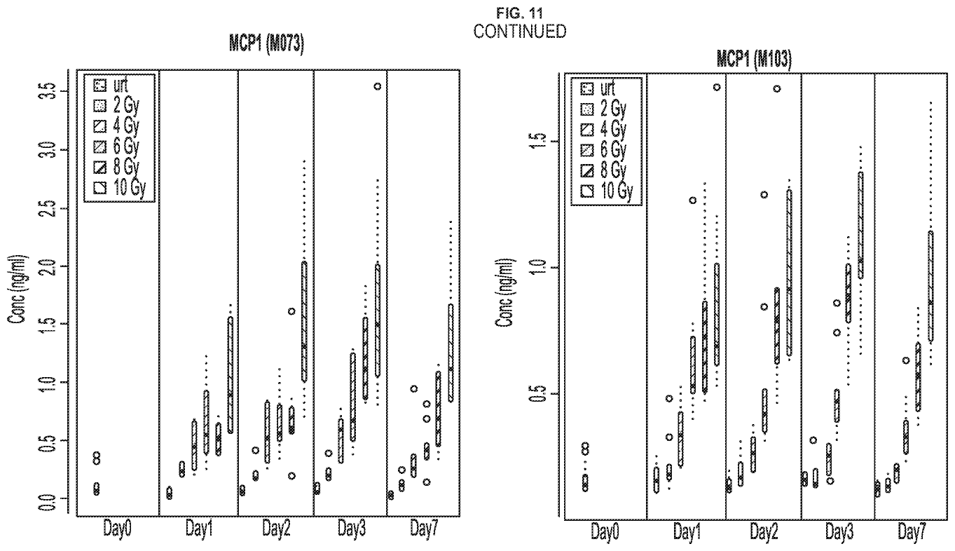

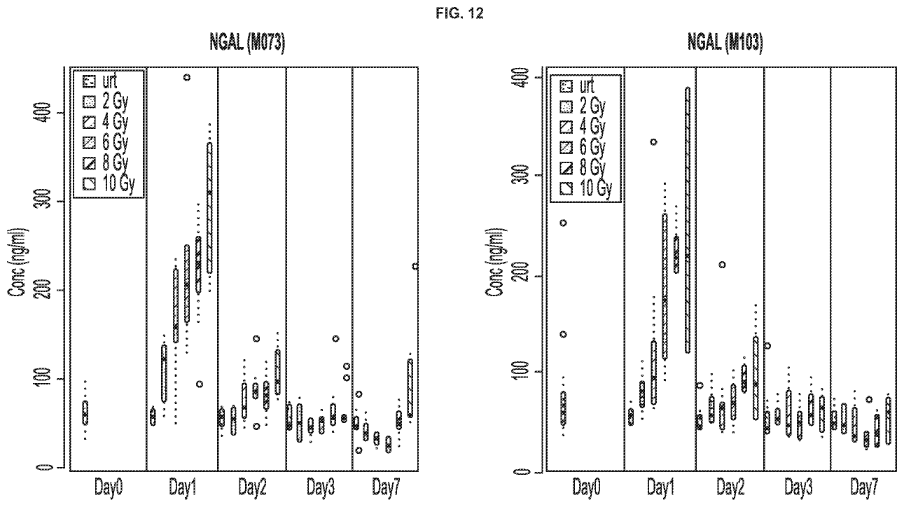

Because the intent was to develop a simple lateral flow assay to measure a panel of radiation responsive proteins, subsequent analysis was focused on protein markers that could be detected at baseline in whole blood with no sample preparation (other than separation of cellular material from plasma, and mixing plasma with buffer before application to the lateral flow test strip). Based on this, candidate proteins for a lateral flow test included AACT, AMY1, FLT3L, NGAL. and MCP1. FIG. 10, FIG. 11, and FIG. 12 show boxplots from the M073 and M103 studies for these 5 proteins. As can be seen from the plots all 5 proteins are strongly upregulated following irradiation in a dose dependent fashion though each follows a different time course.

AACT is elevated at all days post radiation with plasma concentrations that increase with dose but decrease with each day post radiation. Concentrations resulting from exposures of 2 Gy and above are clearly distinguishable from the controls.

AMY1 is significantly elevated on Day 1 post exposure, but is back to baseline by Day 3. AMY1 plasma concentration increases with dose. Concentrations resulting from exposures of 2 Gy and above are clearly distinguishable from the controls.

FLT3L starts to elevate on Day 3 post radiation and increases significantly by Day 7. Plasma concentration increases with dose. Concentrations at all exposures (1 Gy and above) are clearly distinguished from the controls.

NGAL is significantly elevated on Day 1, slightly elevated on Day 3, and nearly back to baseline by Day 5 for lower exposures. Concentration increases with dose. At high exposures (8 and 10 Gy), some elevation is seen out to Day 7 in the M073 data, but not the M103 data. Exposures at 2 Gy and above are clearly distinguished from the controls at Day 1.

MCP1 is significantly elevated at all days post exposure. Concentration increases with dose. Concentrations remain relatively stable out to Day 7 post exposure with perhaps a slight decrease at the low exposures. Concentrations at exposures of 4 Gy and above are clearly distinguished from the controls.

### Fold Change Plots

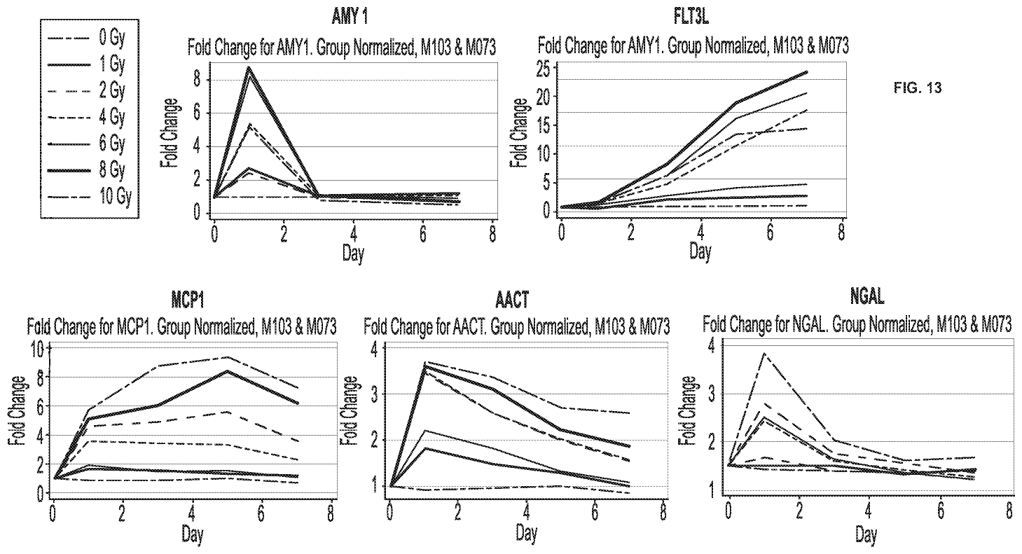

FIG. 13 shows the fold changes observed in the combined M073/M103 NHP studies for AACT, FLT3L, AMY1, NGAL, and MCP1. For each dose group, the means of the Day 0 (pre-radiation) plasma levels were calculated for each protein. The observed plasma levels for each dose group on Days 1, 3, 5, and 7 post-radiation were then normalized to the means of the Day 0 plasma levels for each protein. FLT3L exhibits the highest fold change—about 20 on Day 7 at the highest dose levels. AMY1 and NGAL exhibit their highest fold changes on Day 1 (about 8 and 6, respectively) at the highest dose levels. AACT exhibits a moderate fold change of around 3, peaking on Day 1 and then slowly decreasing with each successive day post exposure. MCP1 shows a peak fold change of around 10 on day 5 for the highest dose levels and a fold change of around 6 on Day 1.

### Classification Analysis

The initial classification analysis focused on the M918 data set and used three different supervised classification algorithms: logistic regression, support vector machine, and conditional inference tree. The approach was to use different combinations of the radiation responsive proteins identified in the immunoassay data to classify the data into one of two absorbed dose groups and identify the best performing combination. For this analysis it was assumed a 4 Gy absorbed dose in an NHP to have approximately the same biological effect of a 2 Gy absorbed dose in a human (based on the estimated LD50 for each species). Thus an absorbed dose of >4 Gy is considered a positive by the classifier and an exposure of <4 Gy is considered a negative. Table 4 shows the classification results for a representative set of high-scoring protein panels for each classifier used. These results shown in the table were obtained by performing 7 iterations of a 5-fold cross validation of the data set. Note that all three classifiers give comparable results. Also, Flt3L and AACT are generally included in the best performing panels; however, a number of other proteins (CRP, IL15, MCP1, NGAL, AMY1A, and HP) can be used interchangeably to yield comparable results. Table 4 indicates Accuracy (Acc), false negative rate (FNR), and false positive rate (FPR) for 12 different protein panels based on 5-fold cross validation. Qualitatively the results are similar in terms of accuracy between the SVM and the logistic regression. Additionally, the same samples are repeatedly misclassified by both statistical learning algorithms. Overall the CI tree performed slightly worse; however, the general ranking of the panels is similar and the misclassified samples tended to be a superset of those misclassified by both logistic regression and SVM.

TABLE 4

| Model | Logistic Regression | | | SVM | | | CI Tree | | |
|---|---|---|---|---|---|---|---|---|---|
| Protein Panel | Acc | FNR | FPR | Acc | FNR | FPR | Acc | FNR | FPR |
| AACT; Flt3L; IL18; NGAL | 93.2% | 4.9% | 8.0% | 93.9% | 6.1% | 6.1% | 89.2% | 12.3% | 9.8% |
| AACT; Flt3L; HPX; NGAL; APOA4 | 95.2% | 3.7% | 5.5% | 93.9% | 5.1% | 6.8% | 89.4% | 12.3% | 9.5% |
| AACT; Flt3L; NGAL; AMY1A | 94.2% | 4.6% | 6.7% | 94.5% | 4.0% | 6.5% | 89.7% | 11.7% | 9.3% |
| AACT; Flt3L; IL15; NGAL; APOA4 | 94.5% | 4.9% | 5.9% | 94.5% | 4.7% | 6.1% | 88.3% | 13.4% | 10.2% |
| AACT; Flt3L; NGAL; APOA4 | 94.1% | 3.8% | 7.3% | 94.8% | 5.0% | 5.3% | 88.7% | 12.2% | 10.7% |
| AACT; Flt3L; NGAL; AMY1A; APOA4 | 94.1% | 4.4% | 6.8% | 95.0% | 3.5% | 6.1% | 90.3% | 10.3% | 9.3% |
| AACT; Flt3L; IL18; NGAL; APOA4 | 93.1% | 6.7% | 7.1% | 94.0% | 4.2% | 7.2% | 88.1% | 12.6% | 11.2% |
| AACT; Flt3L; HPX; NGAL | 94.3% | 5.3% | 5.9% | 93.2% | 3.8% | 8.8% | 89.1% | 12.4% | 9.8% |
| AACT; Flt3L; MCP1; NGAL; APOA4 | 93.6% | 4.2% | 7.9% | 94.2% | 7.7% | 4.6% | 88.6% | 13.6% | 9.6% |
| AACT; Flt3L; IL15; NGAL | 94.5% | 4.6% | 6.1% | 93.9% | 5.3% | 6.6% | 88.9% | 13.7% | 9.4% |
| AACT; Flt3L; NGAL | 94.8% | 4.1% | 6.0% | 93.2% | 3.6% | 8.9% | 88.7% | 12.2% | 10.7% |
| AACT; Flt3L; MCP1; NGAL | 95.2% | 4.4% | 5.0% | 94.1% | 7.1% | 5.1% | 88.3% | 14.2% | 9.7% |

Because the three classification algorithms performed comparably on the M918 data set, the subsequent analyses were performed using logistic regression. Two specific combinations of proteins were focused for analysis: a 4-marker panel consisting of AACT, AMY1, FLT3L, and MCP1, and a 3-marker panel consisting of AMY1, FLT3L, and MCP1. The rationale for considering a 3-marker panel that excludes AACT is that this marker does not appear to be radiation responsive in human radiotherapy patients [Balog et al. , Development of a biodosimeter for radiation triage using novel blood protein biomarker panels in humans and non-human primates, Int. Jour. Radiation Biology, 2019]. These proteins were selected based on the commercial availability of high-quality antibodies suitable for use in a point of care test.

### 2×2 Classification Tables and ROC Curves

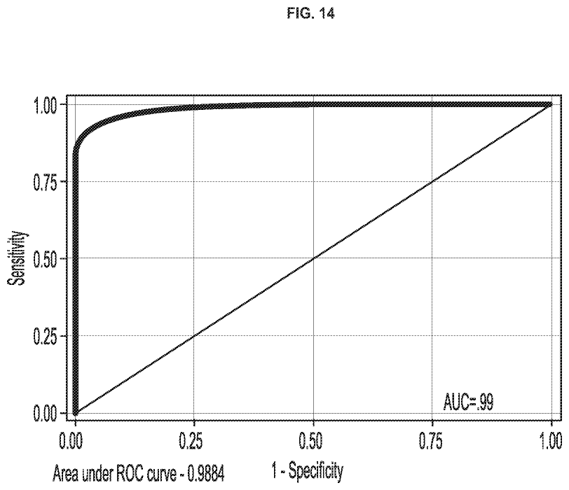

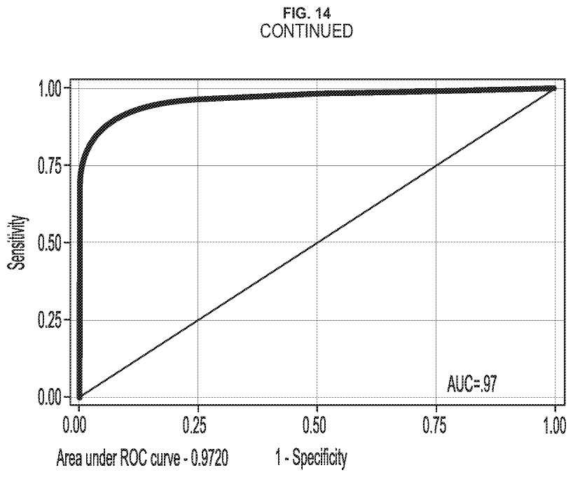

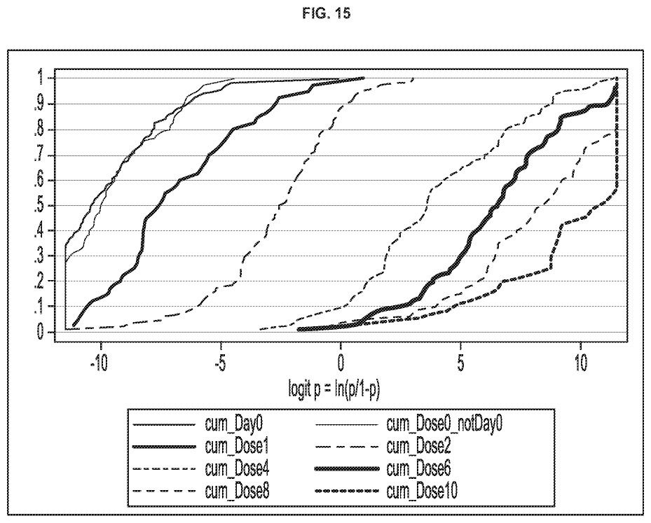

Table 5 summarizes the performance of the LR classifier using either a three- or four-biomarker panel to classify the NHP samples from all three acute studies covering the absorbed dose range of 0 to 10 Gy with collection time points from day 0 (pre-irradiation) to day 7 post-irradiation. In using the classifier, we chose to make no assumptions regarding the dose equivalence between NHPs and humans and therefore used 2 Gy as the classifier cutoff—samples from subjects receiving absorbed doses of >2 Gy were considered as positives and those below 2 Gy were considered as negatives. From the tables, we can see that the two panels perform comparably. The 3-marker panel achieves an overall sensitivity and specificity of 94% and 90% respectively, corresponding to a false negative rate (FNR) and a false positive rate (FPR) of 6% and 10% respectively. The 4-marker panel performs slightly better with a sensitivity and specificity of 95% and 92% respectively, corresponding to an FNR and FPR of 5% and 8% respectively. The corresponding Receiver Operating Characteristic (ROC) curves for both panels are shown in FIG. 14 and have AUCs of around 0.98. AnROC curve is a plot that depicts the trade-off between sensitivity and (1-specificity) across a series of cut-off points for the Principal Component Analysis (PCA) test statistic. Table 5 indicates classification results obtained from all Day 0 to Day 7, 0 Gy to 10 Gy NHP samples using the three- and four biomarker panels. The sensitivity and specificity are 92.4% and 94.4% respectively for 3 biomarkers and 94.4% and 93.7% for 4 biomarkers. Because one sample was not measured for MCP1, the total number of samples classified was 894.

TABLE 5

| | |
|---|---|
| Condition Negative | Condition Positive |

3 Marker panel (AMY1, FLT3L, MCP1)

| | | |
|---|---|---|
| Predicted Negative | 354 | 39 |
| Predicted Positive | 26 | 475 |
| Total | 380 | 514 |

4 Marker panel (AACT, AMY1, FLT3L, MCP1)

| | | |
|---|---|---|
| Predicted Negative | 356 | 29 |
| Predicted Positive | 24 | 485 |
| Total | 380 | 514 |

### Classification Results by Dose and Day

Table 6 and Table 7 show, for each dose and day post exposure, the percentage of observations that were classified as positives using either our 3-biomarker or 4-biomarker panels. Also listed are the number of observations (subjects) for each dose and time point. Note that for absorbed doses >2 Gy, the true positive rate is high, ranging from >75% at 2 Gy and increasing to >94% at higher absorbed doses. Table 6 indicates NHP classification results using the three-biomarker panel for each dose/day subgroup. As can be observed within the red box, at absorbed doses >2 Gy and beyond day 0, a large percentage of the observations are called as positives.

TABLE 6

| | Dose (Gy) | | | | | | | | | | | | | |
|---|---|---|---|---|---|---|---|---|---|---|---|---|---|---|
| | 0 | | 1 | | 2 | | 4 | | 6 | | 8 | | 10 | |
| Day | N | % | N | % | N | % | N | % | N | % | N | % | N | % |
| 0 | 180 | 4% | | | | | | | | | | | | |
| 1 | 30 | 3% | 20 | 25% | 30 | 67% | 30 | 87% | 20 | 100% | 30 | 100% | 19 | 100% |
| 2 | 10 | 0% | 10 | 10% | 10 | 30% | 10 | 70% | | | 10 | 100% | 9 | 89% |
| 3 | 30 | 7% | 20 | 20% | 30 | 80% | 30 | 100% | 20 | 100% | 30 | 97% | 19 | 100% |
| 4 | | | | | | | | | | | | | | |
| 5 | 20 | 0% | 10 | 30% | 20 | 100% | 20 | 100% | 20 | 100% | 20 | 100% | 9 | 100% |
| 7 | 30 | 3% | 20 | 10% | 30 | 77% | 30 | 100% | 20 | 100% | 30 | 100% | 18 | 100% |
| Total N | 300 | | 80 | | 120 | | 120 | | 80 | | 120 | | 74 | |
| % Positive | | 4% | | 19% | | 75% | | 94% | | 100% | | 99% | | 99% |

Table 7 indicates NHP classification results using the four-biomarker panel for each dose/day subgroup. As can be observed within the red box, at absorbed doses >2 Gy and beyond day 0, a large percentage of the observations are called as positives.

TABLE 7
% Positive (NHPs)

| | Dose (Gy) | | | | | | | | | | | | | |
|---|---|---|---|---|---|---|---|---|---|---|---|---|---|---|
| | 0 | | 1 | | 2 | | 4 | | 6 | | 8 | | 10 | |
| Day | N | % | N | % | N | % | N | % | N | % | N | % | N | % |
| 0 | 180 | 3% | | | | | | | | | | | | |
| 1 | 30 | 3% | 20 | 10% | 30 | 73% | 30 | 93% | 20 | 100% | 30 | 100% | 19 | 100% |
| 2 | 10 | 0% | 10 | 30% | 10 | 60% | 10 | 100% | | | 10 | 100% | 9 | 100% |
| 3 | 30 | 3% | 20 | 40% | 30 | 90% | 30 | 100% | 20 | 100% | 30 | 100% | 19 | 100% |
| 4 | | | | | | | | | | | | | | |
| 5 | 20 | 5% | 10 | 20% | 20 | 95% | 20 | 100% | 20 | 100% | 20 | 100% | 9 | 100% |
| 7 | 30 | 3% | 20 | 0% | 30 | 63% | 30 | 100% | 20 | 100% | 30 | 100% | 18 | 100% |
| Total N | 300 | | 80 | | 120 | | 120 | | 80 | | 120 | | 74 | |
| % Positive | | 3% | | 19% | | 77% | | 98% | | 100% | | 100% | | 100% |

### Graphs of Estimated Probability of Exposure for NHP

Logistic regression was used on NHP data to estimate the probability of exposure for each observation and plot the cumulative distribution functions by dose for the 3-biomarker panel in FIG. 15. Biomarkers were first log transformed and then normalized by dividing by the average transformed baseline value (separately by study). Exposure was defined as a cumulative dose of >2.0 Gy. Cumulative distribution functions of the logit of the estimated probability of exposure were then graphed for groups of observations with same cumulative dose. As can be seen in FIG. 8, with increasing exposure the CDFs shift to the right of the graph. The curves for absorbed doses >2 Gy are clearly distinguishable from the curves for the baseline (0 Gy) animals indicating the utility of the panel of only 3 biomarkers in correctly classifying samples into one of two absorbed dose groups.

### Animal Studies

A radiation biodosimeter that can be used at the point of need to triage individuals potentially exposed to ionizing radiation would have significant impact on the ability to provide timely and effective medical treatment and enable efficient use of scarce medical resources following a major nuclear event. Such a device must be capable of rapid detection of a panel of biomarkers that are indicative of absorbed radiation dose and provide a qualitative assessment of whether the individual received an absorbed dose of >2.0 Gy. Because there is limited data on the radiation response of healthy humans, and it is unethical to conduct such studies, results are presented of three large non-human primate irradiation studies in an effort to identify suitable panels of protein biomarkers for radiation biodosimetry that may be detected in a small blood sample that can be collected non-invasively. In these studies, used mass spectrometry was used to identify at least 30 proteins that change significantly following radiation exposure. Various subsets of these proteins can accurately classify an NHP data set consisting of 894 samples covering the 0-10 Gy absorbed dose range and day 1-7 post exposure time points. Specific panels of 3 or 4 biomarkers were identified, detected using immunoassay, that can be used in a point of need biodosimeter to accurately classify an unknown sample into two absorbed dose groups.

### Human Clinical Studies

All human samples used were obtained with informed consent under an appropriate IRB approved protocol. Human radiotherapy patient samples were obtained at the Stanford University Medical Center (SUMC). In addition, samples from several special population groups were also obtained at Stanford including individuals with trauma and infections as well as from healthy donors. Samples from several special population groups including individuals with obesity, diabetes, rheumatoid arthritis, compromised immune systems, and pregnancy as well as samples from healthy donors were obtained commercially from Bioreclamation. Burn patient samples were obtained at the UC Davis Medical Center (UCDMC) and immune compromised samples were obtained from the Duke University Medical Center.

### Radiotherapy Patients

These patients were typically between 18 and 65 years old, and were primarily undergoing treatment for leukemia or lymphoma. Patients were excluded from the study if they had received any chemotherapy within 21 days prior to radiation treatment, or had received any prior radiation treatment. The most common treatment plan for TBI patients used at the SUMC includes three doses of 120 cGy on days 1-3 and 2 doses on day 4, with each dose after the first dose for a day separated by 3 hours. TBI was delivered with 15-MV photons with 2 equally weighted beams (anterior-posterior/posterior-anterior) at a dosage rate of 0.13-0.17 Gy/min. Custom-tailored blocks were designed for each patient to shield the lungs. A total of 232 samples were collected from 65 patients. TBI samples were collected from all 65 patients (35M/30F) pre-treatment on day 1, from 60 patients (32M/28F) pre-treatment on day 2 (after 3 fractions), from 60 patients (31M/29F) on day 3 (after 6 fractions) and from 47 patients (24M/23F) on day 4 (after 9 fractions) corresponding to cumulative total absorbed doses of 0, 3.6, 7.2, and 10.8 Gy.

### Control and Special Population Groups

Samples were collected from both control and special population groups. The control group consisted of 272 (155M/117F) samples from healthy donors and included samples from 154 adult (age range 22-65), 61 adolescents (age range 12-21), and 57 geriatric (age range >65) individuals. These samples were obtained from both SUMC and Bioreclamation and covered a demographic distribution representative of the US.

Additional blood samples were purchased from Bioreclamation and included 96 (50M/50F) type II diabetics, 88 (50M/38F) obese (BMI>30), 100 pregnant, and 89 (44M/45F) rheumatoid arthritis patients. Blood samples from 53 (39M/14F) individuals experiencing trauma and 61 (19M/42F) individuals with mild infections, were collected by SUMC. These individuals typically experienced bone breaks, lacerations, knife and bullet wounds or had upper respiratory infections. Samples from 12 immune compromised individuals (CD4 counts <200) were obtained from both Bioreclamation and Duke. A total of 48 samples were obtained from 10 (9M/1F) burn patients collected at multiple time points over a period of 1 to 7 days following admission to the UCDMC. Burn patients were included in the study provided they were 18 years or older, had no admission diagnosis other than burn injury, and had a burn injury that included greater than or equal to 10% of total body surface area but less than or equal to 30%.

### Blood Collection

Venous blood was collected using a single BDTM P100 Blood Collection System for preservation of plasma proteins. Tubes were collected to the full 8 mL volume and each was inverted 8-10 times to thoroughly mix the P100 anticoagulant and then placed inside a ziplock bag on a layer of wet ice inside a Styrofoam container. Each P100 tube containing blood was centrifuged at 1600 g for 30 min. Using a 1000 μl micro pipettor with appropriately sized tips, 500 ill aliquots of plasma were transferred from the top layer in the P100 tubes into the appropriate number of individual screw-cap 1.5-mL microcentrifuge tubes. These aliquot tubes were stored at −80° C. until shipment on dry ice to SRI. All received samples were stored at −80° C. until analysis by mass spectroscopy or immunoassay.

### NHP Studies

NHP samples were obtained from several different irradiation studies as described in more detail elsewhere [Balog et al. Development of a point-of-care radiation biodosimeter: studies using novel protein biomarker panels in non-human primates, Int. Jour. Radiation Biology, 2019]. These consisted of three large TBI acute exposure studies performed at CioxLab (Montreal, Canada) and LBERI (Albuquerque, N. Mex.). The total NHP acute exposure sample set obtained from all three studies consists of 895 samples from normal (baseline) NHPs as well as NHPs receiving absorbed doses of radiation in the range of 1 to 10 Gy with blood collections in the 1 to 7-day post-irradiation time window. Each dose group contained 10 (5M/5F) animals (ages ˜4 yrs). The use of animals and study protocols were approved by the Institutional Animal Care and Use Committee (IACUC) in all participating institutes and by the sponsor.

In addition to these acute irradiation studies two other NHP irradiation studies were conducted at LBERI that included both acute and fractionated exposures. In these studies, animals received either acute, double, or triple fractionated doses of 6 MV X-rays from a Varian 600c LINAC at a dose rate of 50-80 cGy/min. Single acute dose animals received irradiation with a bilateral scheme that delivered half of the dose to each of the left and right lateral sides. Fractionated dose animals received each fraction to a single side of the animal with the side alternated between doses.

Two fractionated dosing schemes were used, chosen to mimic irradiation protocols commonly used for human patients receiving TBI therapy. The first scheme consisted of administering two 1.5 Gy dose fractions per day. The second scheme consisted of administering three 1.2 Gy dose fractions per day. These schemes were applied in two studies. In the first study the dose fractions were administered on four consecutive days beginning on day 0 for total cumulative doses of either 3, 6, 9, and 12 Gy or 3.6, 7.2, 10.8, and 13.2 Gy on days 1, 2, 3, and 4 for the double and triple fractionated dose schemes respectively. In the second study the dose fractions were administered only on day 0 for total cumulative doses of either 3 or 3.6 Gy for each scheme respectively. The fractionated dose groups consisted of 12 animals (6M/6F) in the first study and 8 animals (4M/4F) in the second study. The acute exposure groups in the first study consisted of 3 animals (2F/1M) receiving a single dose of 12 Gy and 4 animals (2M/2F) receiving a single dose of 13.2 Gy. In the second study, two groups of 8 animals (4M/4F) received a single acute dose of either 3 Gy or 3.6 Gy. In both studies, blood samples were collected from each animal pre-irradiation on day −3, and prior to irradiation on days 1, 2, 3, 4, and 7.

### Immunoassays

Immunoassays were performed in duplicate using conventional ELISA performed in a sandwich format. ELISA assays were performed on the human samples for 6 different protein targets using commercially available kits. These targets included AACT, AMY1, FLT3L, IL15, MCP1, and NGAL. Each assay plate included one or more plasma sample standards to evaluate assay variability. For all assays, the inter-plate CVs ranged from 2.3% to 14%.

As described elsewhere [Balog et al. Development of a biodosimeter for radiation triage using novel blood protein biomarker panels in humans and non-human primates, Int. Jour. Radiation Biology, 2019], immunoassays conducted on the NHP samples were performed in duplicate utilizing conventional ELISA in a sandwich format. ELISA assays were performed for the same 6 protein targets as for the human samples. Each assay plate included one or more plasma sample standards to evaluate assay variability. For all NHP assays, the inter-plate CVs ranged from 3.6% to 11.7%, with an average CV of 10%.

### Statistical Methods

Data analysis was performed using several different analysis packages: the comprehensive statistical analysis package known as R, which is available as freeware and widely used within the biostatistics community, the Matlab Statistics toolpack, and the Stata statistical and data analysis software. Initial data processing consisted of reading in the raw data files produced by the ELISA instrument and preparing a master data file consisting of Excel spreadsheets of the data for each protein for each plasma sample. Standard analyses included preparation of boxplots, histograms, assay CVs, correlation tables, and fold-change plots for each protein. Most analyses were performed on log-transformed data as we found the transformed data to be more normally distributed than the untransformed data. Both paired and unpaired t-tests were performed as well as linear regressions to identify proteins that change significantly from baseline as a result of irradiation. Data sets were classified using several supervised classifiers that included logistic regression, support vector machine, and conditional inference trees as well as the described classification methods.

The described classification methods compares the biomarker concentration from an unknown sample against the distribution of concentrations for normal individuals for that biomarker. The result of this comparison is a value p which is the proportion of normal healthy subjects that have a biomarker concentration greater than that measured in the unknown sample. This value is referred to as the “upper tail probability” for the biomarker of the unknown sample. This process is repeated for all biomarkers in a panel and a test statistic (TS) is obtained by summing −ln(p) for each biomarker. The TS value obtained from the unknown sample is then compared against a threshold value to determine whether the test result is positive or negative. The threshold value for the TS is obtained from observations on normal individuals who have not been exposed to radiation and was set to yield a false-positive rate of 5% (this can be varied to trade FPR for FNR). This approach is similar to Fisher's method of combining probabilities [Fisher, Statistical Methods for Research Workers. Oliver and Boyd (Edinburgh). ISBN 0-05-002170-2, 1925] and offers several advantages for our application using a panel of biomarkers: (1) it is based only on the distribution of normals so no data from irradiated individuals or animals is required, (2) it is scalable from a single biomarker to many biomarkers, (3) for normalized and standardized data sets, the algorithm is species independent for humans and NHPs (after scaling by each species' baseline protein concentrations, an algorithm developed on humans can be applied to NHP and vice versa), and (4) it makes no assumptions regarding dose equivalence between humans and NHPs. In our analyses, all data are first log transformed and then the mean and standard deviation for each transformed protein is calculated for normal healthy subjects. Our data sets are then standardized by subtracting the mean concentration of normals from each measured value for each protein and dividing by the standard deviation of normals. This procedure is performed separately for each species.

### Human Studies Results

### Immunoassay

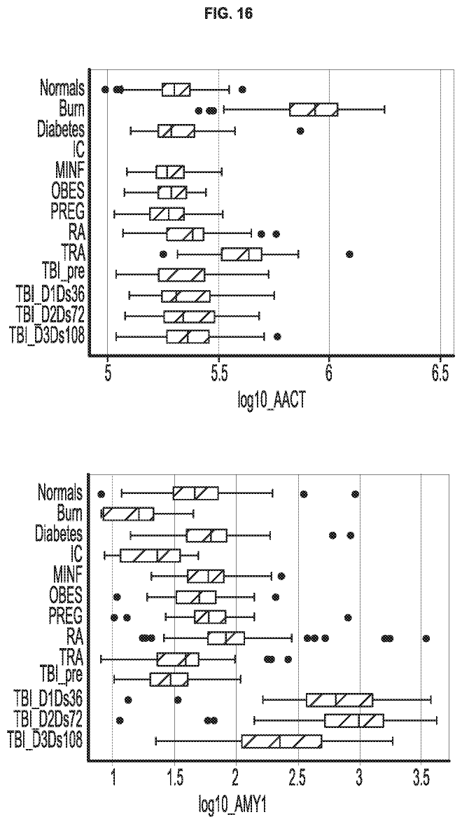

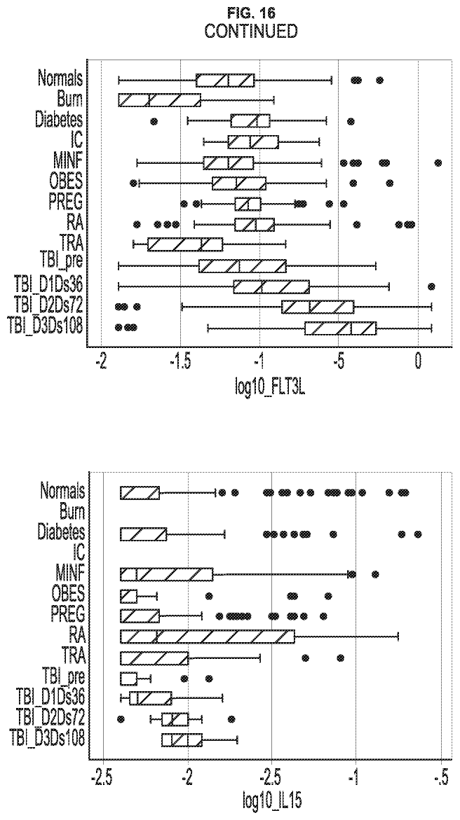

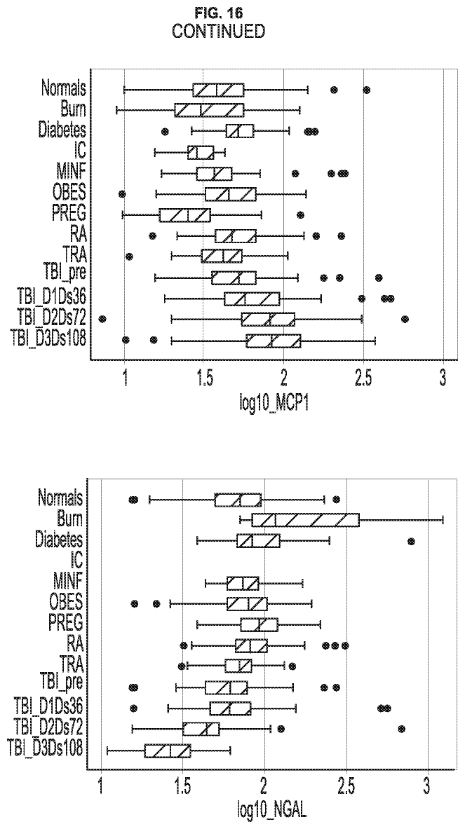

FIG. 16 shows the boxplots for the human data sets for the proteins AACT, AMY1, FLt3L, IL15, MCP, and NGAL for the control, special population, and TBI groups (for non-standardized or normalized data). Due to the relatively large variation in protein concentrations, the log 10 of the protein concentration (in ng/ml) is plotted.

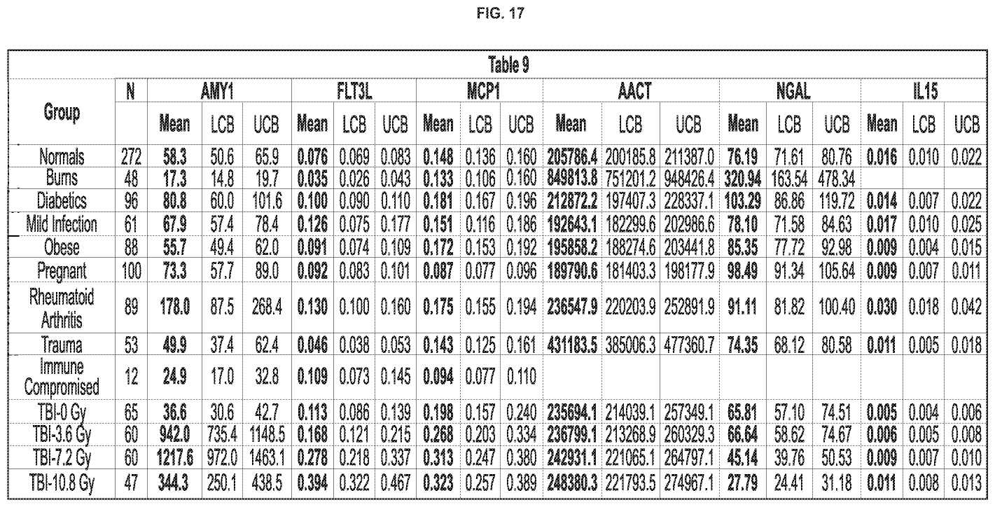

T-test comparisons were performed of the various human confounder groups against the control group using the ELISA data obtained from analysis of human plasma samples (Table 8). Table 8 shows for each group whether a protein of interest is higher (down-arrow) or lower (up-arrow) as compared to the controls. A down or up arrow indicates that the p-value is less than 0.05 and therefore likely to be statistically significant. To correct for multiple comparisons, we applied the Bonferroni correction factor to each p-value to ensure that there is no more than a 5% probability that there are any false statistically significant results across all tests. Although for any given protein, there are differences between some of the special population groups and controls, none of the special groups exhibit a pattern for the 5 proteins of interest (AACT, AMY1, FLT3L, NGAL, MCP1) that is similar to that observed in the radiotherapy patients. As shown in FIG. 17, Table 9 lists the mean plasma concentrations (in ng/ml) and the 95% confidence intervals for each human subgroup for the proteins AMY1, Flt3L, MCP1, AACT, NGAL, and IL15 as measured by ELISA. The red boxes in the table highlight the mean concentrations observed in the human radiotherapy patients for each biomarker. Note that for the markers AMY1, FLT3L and MCP1 we observe mean values that increase with absorbed radiation dose in these patients. No significant change is observed for AACT, NGAL levels are observed to drop, and the boxplot for IL15 shows a slight increase with increasing absorbed dose.

For AACT the boxplot in FIG. 16 shows that this protein does not appear to be radiation responsive in human TBI patients as no significant change in the levels of this protein are observed compared to controls. This is distinctly different from what is observed in NHPs where AACT appears to be strongly radiation responsive and increases with increasing absorbed dose [Balog et al. Development of a point-of-care radiation biodosimeter: studies using novel protein biomarker panels in non-human primates, Int. Jour. Radiation Biology, 2019]. It is at present not clear why the radiation response of AACT is so different between the human TBI patients and NHPs. AACT levels in the trauma and burn patient groups appear to be increased relative to the control group. The t-test results in Table 8 and the mean plasma concentrations in Table 9 reflect these results. Levels of AACT in the TBI patients are not statistically different from levels in the control group. The trauma and burn subgroups differ significantly from the control group both exhibiting exceedingly low p-values.

AMY1 levels in the TBI patient subgroup shows strong radiation response with mean levels significantly increased relative to both controls as well as the same subjects prior to radiation exposure. The AMY1 levels post-exposure do not appear to depend strongly on cumulative radiation dose—they increase significantly above baseline (p-values<0.0001) and appear to drop at the highest dose (although still well above baseline). The t-test results in Table 8 show that several other subgroups show mild elevations in AMY1 levels relative to the controls. These include pregnant and rheumatoid arthritis individuals. However, as can be seen in the boxplot and in Table 9, the elevations in these subgroups are far below what is observed in the radiotherapy patients post exposure. AMY1 levels in both the burn patients and in the TBI patients pre-exposure are lower than that observed in the controls.

The FLT3L boxplots show TBI patients post-exposure have plasma levels that are significantly more elevated than the controls and the levels increase with increasing dose and timepoint following exposure. Excluding the diabetic, rheumatoid arthritic, burn and trauma groups, the other subgroups have levels that are comparable to the controls. This is also reflected on the t-test table. The post-exposure TBI patients all exhibit FLT3L levels that are elevated relative to the controls with high statistical confidence (p-values<0.0001). The lower levels of FLT3L observed in the burn and trauma patients appear to be statistically significant (p-value<0.0001). The elevated levels of FLT3L observed in the diabetic and arthritic individuals are well below that observed in the post exposure TBI patients as can be seen from Table 9.

MCP1 levels exhibit a moderate increase with absorbed dose in the TBI patients and are statistically distinguishable from levels in the control group (p-values<0.0001). With the exception of the diabetic, pregnant, and rheumatoid arthritic subgroups, all other subgroups exhibit levels that are comparable with and statistically indistinguishable from the control group. As can be seen from Table 8, for the diabetic and rheumatoid arthritic subgroups, the observed elevation in MCP1 levels are well below that observed in the TBI patients. The pregnant subgroup shows levels that are below that observed in the controls. Although the pre-radiation TBI patients show a significant elevation relative to the controls (p-value=0.03), as can be seen from Table 9, the mean plasma concentration for this subgroup is well below that observed in the same individuals post exposure.

Although the data for IL15 is limited to only about half the number of individuals in each subgroup (and missing for the burn and IC subgroups), the boxplot shows that plasma levels increase slightly for the TBI patients post exposure. However, the t-test results indicate that there is no statistically significant difference between the TBI post exposure subgroups and the controls. As is the case for AACT, this result is distinctly different from what is observed in NHPs where IL15 is strongly radiation responsive and increases with increasing absorbed dose [Balog et. al., to be published]. It is not clear why the radiation response of this marker is different in humans and NHPs.

For NGAL the boxplot shows that plasma levels of this protein appear to decrease with radiation exposure. The t-test results confirm this and also show that several of the special population subgroups show significant differences relative to the controls. The burn, diabetic, pregnant, and RA subgroups all exhibit levels of NGAL that are elevated relative to the controls.

From these results, and in particular from Table 2, the three proteins AMY1, FLT3L, and MCP1 provide a unique descriptor for human TBI patients (and by extrapolation, to TBI exposed normal healthy humans). All three are significantly elevated in response to absorbed doses of radiation, and with the exception of the RA patients, no other human subgroup studied exhibits a similar behavior. For the RA patients where all of these markers appear to be elevated relative to controls, as we will see below, our classifier exhibits a higher false positive rate with these subjects. Also, as discussed below, inclusion of either IL15 or NGAL or both in our panel does not improve our classification results.

Table 8 indicates t-test results from the human data sets. Arrows indicate the resulting p-value was statistically significant (<0.05). Up and down arrows indicate that a protein levels are higher or lower relative to the control group. The numbers in parenthesis are the resulting p-values. The results were obtained on log 10 transformed data using the Bonferroni correction for multiple comparisons. The TBI results are separated into subsets denoted by time point and total dose. For example, d1ds360 means day 1 samples with total cumulative dose of 360 cGy.

TABLE 8
Ttest(log10 Bonferroni adjustment on just CTRL vs others; pool.sd = var.eq = F)

| GROUP vs CTRL | AACT | AMY1 | FLT3L | MCP1 | NGAL | IL15 |
|---|---|---|---|---|---|---|
| BURN | ↑(<1e−4) | ↓(<1e−4) | ↓(<1e−4) | — | ↑(.007) | |
| DIEB | — | — | ↑(<1e−4) | ↑(<1e−4) | ↑(<1e−4) | — |
| MINF | — | — | — | — | — | — |
| OBES | — | — | — | — | — | — |
| PREG | — | ↑(.002) | ↑(<1e−4) | ↓(<1e−4) | ↑(<1e−4) | — |
| RA | — | ↑(<1e−4) | ↑(<1e−4) | ↑(.005) | ↑(.009) | — |
| TRA | ↑(<1e−4) | — | ↓(<1e−4) | — | — | — |
| TBI-Pre | — | ↓(<1e−4) | — | ↑(.03) | — | — |
| TBI-D1Ds3.6 | — | ↑(<1e−4) | ↑(<1e−4) | ↑(<1e−4) | — | — |
| TBI-D2Ds7.2 | — | ↑(<1e−4) | ↑(<1e−4) | ↑(<1e−4) | ↓(<1e−4) | — |
| TBI-D3Ds10.8 | — | ↑(<1e−4) | ↑(<1e−4) | ↑(<1e−4) | ↓(<1e−4) | — |

Table 9, shown in FIG. 17, indicates Mean, upper and lower 95% confidence bounds for biomarker concentrations (in ng/ml) for each human subgroup. N is the number of subjects in each group*. The red boxes highlight the mean values observed in human radiotherapy patients.

*AACT, NGAL, and IL15 data are unavailable for the immune compromised patients. IL15 values are not available for the burn patients and are only available for about half of the individuals in each other subgroup

### Comparison of Human and NHP Data

### Fold Change Comparison for Similar Fractionated Dosing

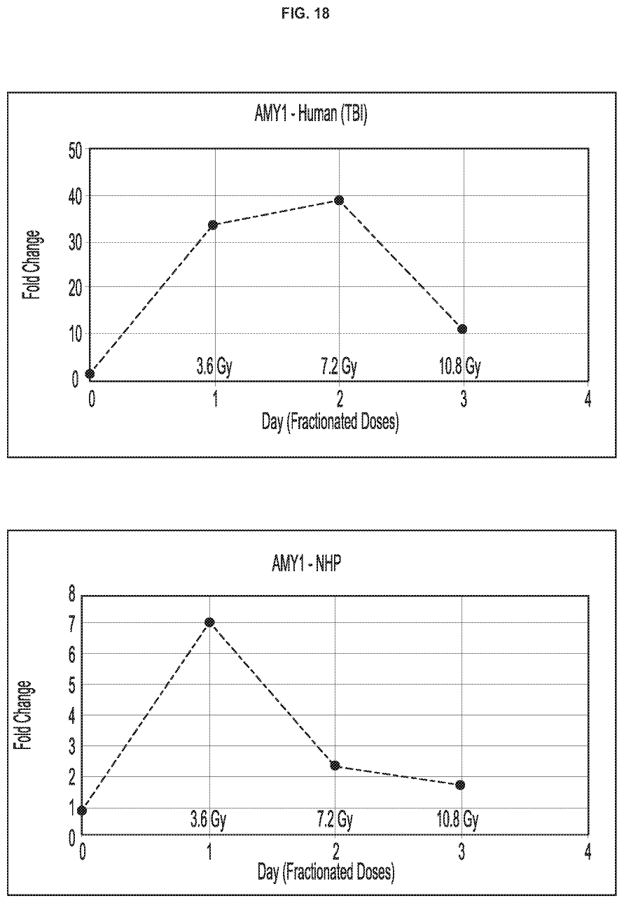

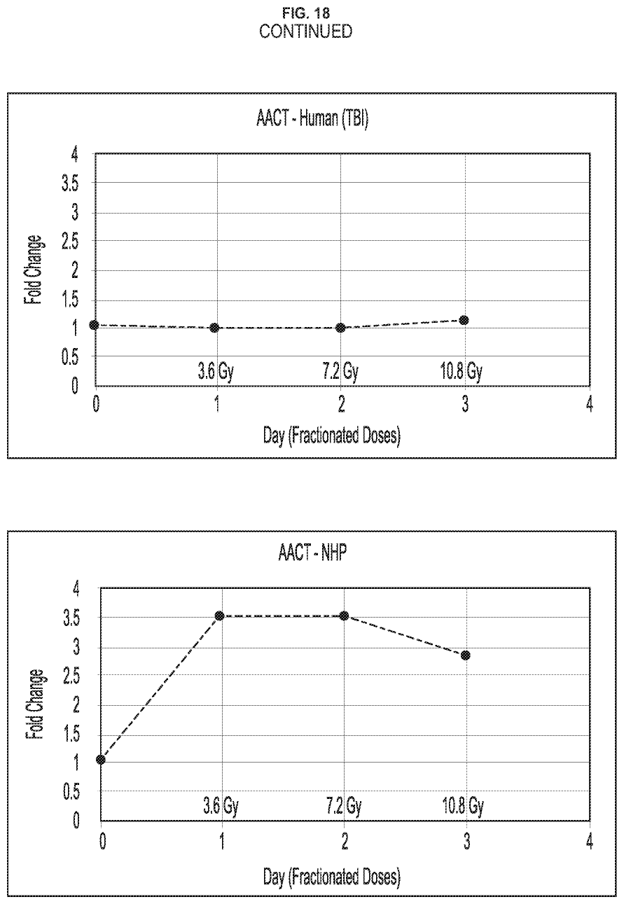

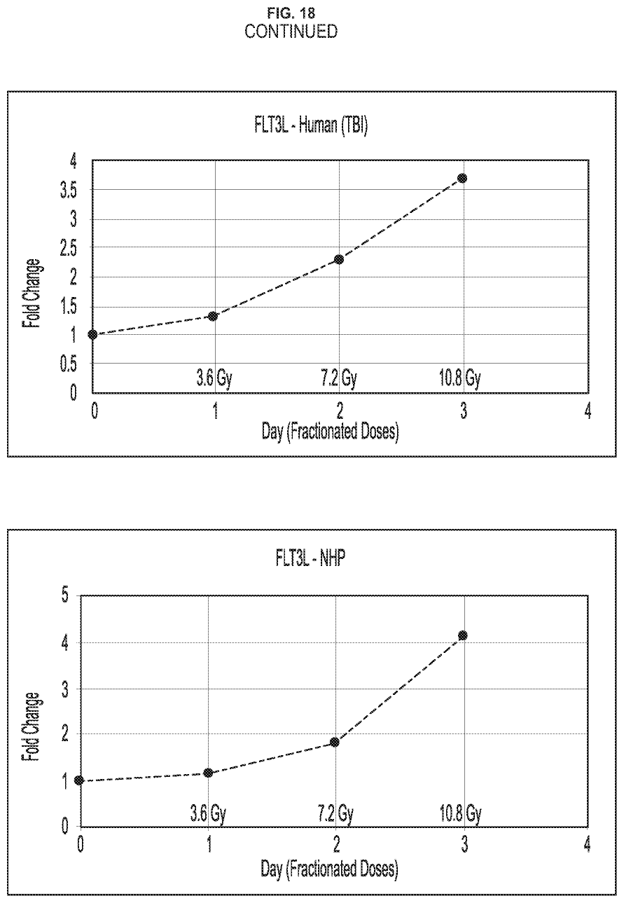

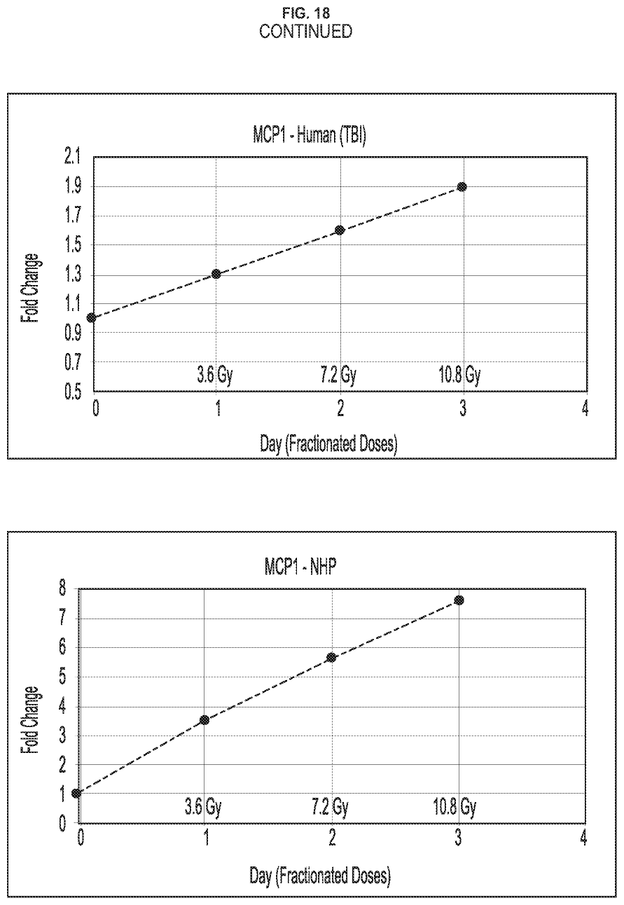

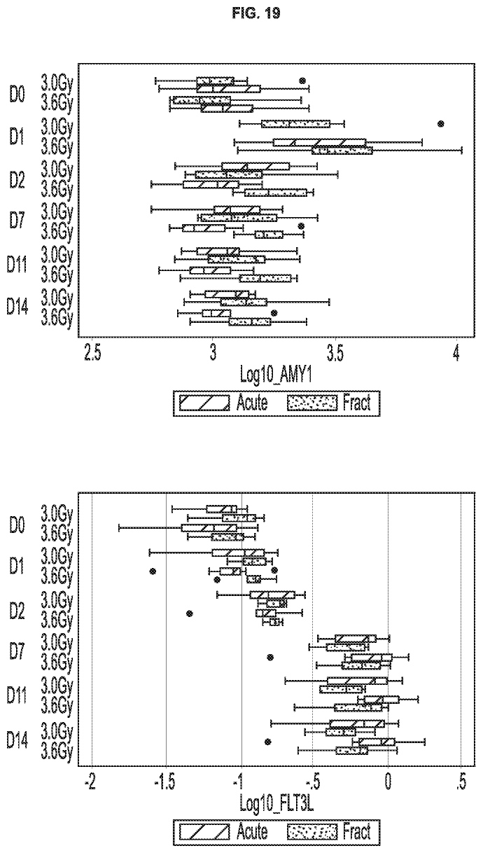

The plasma concentrations of the 3 biomarkers AMY1, FLT3L, and MCP1 are up-regulated in both humans and NHPs in response to ionizing radiation and generally increase with increasing cumulative absorbed dose. As mentioned previously, AACT is up-regulated in response to radiation in NHPs but not in human TBI patients. FIG. 18 compares the fold changes measured in human TBI patients and NHPs for these four proteins. The NHP fold changes were obtained from a study where the NHPs received the same fractionated dosing as the human TBI patients—namely 3×1.2 Gy fractions per day for three consecutive days with sample collections on days 1, 2, and 3 following cumulative absorbed doses of 3.6, 7.2, and 10.8 Gy respectively. As can be seen, for similar fractionated dosing protocols, the fold change patterns are similar in both species, although the magnitudes of the fold changes are different, particularly for AMY1.

### NHP Acute/Fractionated Dosing Comparison

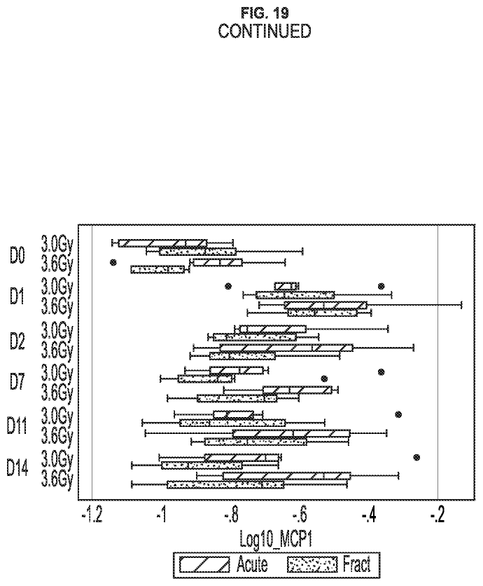

A comparison of NHP acute versus fractionated dosing indicates that for the same cumulative dose, administered on the same day, a fractionated dose is comparable to an acute dose in elevating the biomarkers of interest, as shown in FIG. 19. T-test comparisons between the fractionated and acute dosing protocols on each day result in p-values that are not statistically significant. A permutation test, in which 1000 iterations of assigning animals randomly to various exposure groups, was also performed that confirmed the results of the t-test comparisons. However, given that there were only 8 animals (4M/4F) in each dose group, this result should be considered preliminary. Future studies comparing fractionated and acute dosing protocols should contain additional animals for improved statistical power.

### Classification of Human Data Sets

For human subjects including normals, special populations, and TBI patients, Table 10 shows the percentage of observations that were classified as positive using our 3-biomarker panel and percentile classification algorithm. For normal baseline humans, the false positive rate was 4.8%. For all unexposed humans, the overall false positive rate was 8.8%. Observed error rates were slightly higher than baseline rates for individuals with diabetes (9.4%) and obesity (6.8%) and significantly higher for rheumatoid arthritis (21.3%) and mild infection (13.1%) patients. For non-exposed TBI patients, the false positive rate was 9.2%. For radiation-exposed TBI patients, the false negative rates were 10%, 5%, and 15% for individuals who received cumulative fractionated doses of 3.6, 7.2, and 10.8 Gy, respectively.

It is important to note that inclusion of either IL15 or NGAL or both in our panel does not improve classification accuracy, though it does reduce the sample set by approximately half due to the fact that IL15 was measured on about half of human samples. Inclusion of both in our panel results in a reduced data set of 508 samples, and AUC of 0.995 an FPR of 6.7% and an FNR of 0%. Classification of the same 508 sample set with our 3 marker panel results in an AUC of 0.997, an FPR of 5.9% and an FNR of 0%.

Table 10 indicates classification summary for all human subgroups using the three-biomarker panel. At absorbed TBI doses >3.6 Gy, a large percentage of the observations are called as positives. N is the total number of subjects in each subgroup with the exception of the burn patients where 48 samples were obtained from 10 burn patients.

TABLE 10
% Positive (Humans)

| | Dose (Gy) | | | | | | | |
|---|---|---|---|---|---|---|---|---|
| | | | 3.6 | | 7.2 | | 10.8 | |
| | 0 | | (Day 1) | | (Day 2) | | (Day 3) | |
| Group | N | % | N | % | N | % | N | % |
| Normals | 272 | 4.8% | | | | | | |
| Burn | 48 | 0.0% | | | | | | |
| Diabetics | 96 | 9.4% | | | | | | |
| Mild Infection | 61 | 13.1% | | | | | | |
| Obese | 88 | 6.8% | | | | | | |
| Pregnant | 100 | 4.0% | | | | | | |
| Rheumatoid Arthritis | 89 | 21.3% | | | | | | |
| Trauma | 53 | 0.0% | | | | | | |
| Immune Compromised | 12 | 0.0% | | | | | | |
| TBI-fract | 65 | 9.2% | 60 | 90% | 60 | 95% | 47 | 85% |
| Total N | 884 | | 60 | | 60 | | 47 | |
| % Positive | | 7.4% | | 90% | | 95% | | 85% |

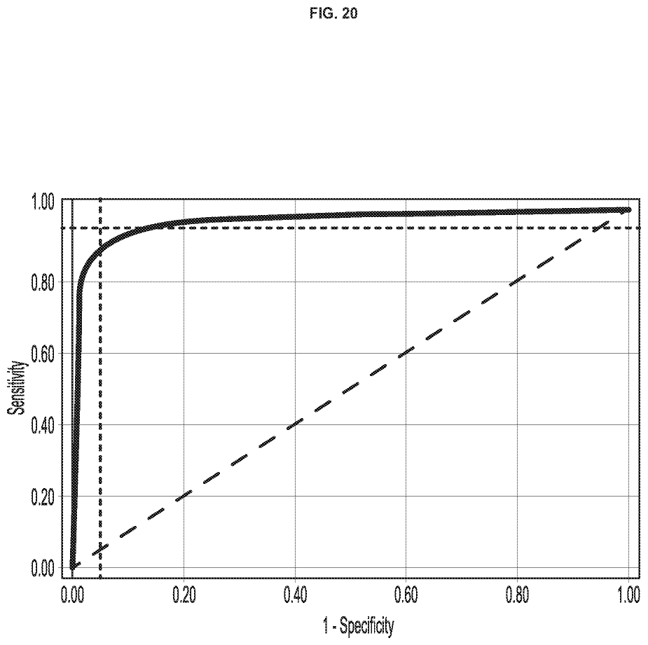

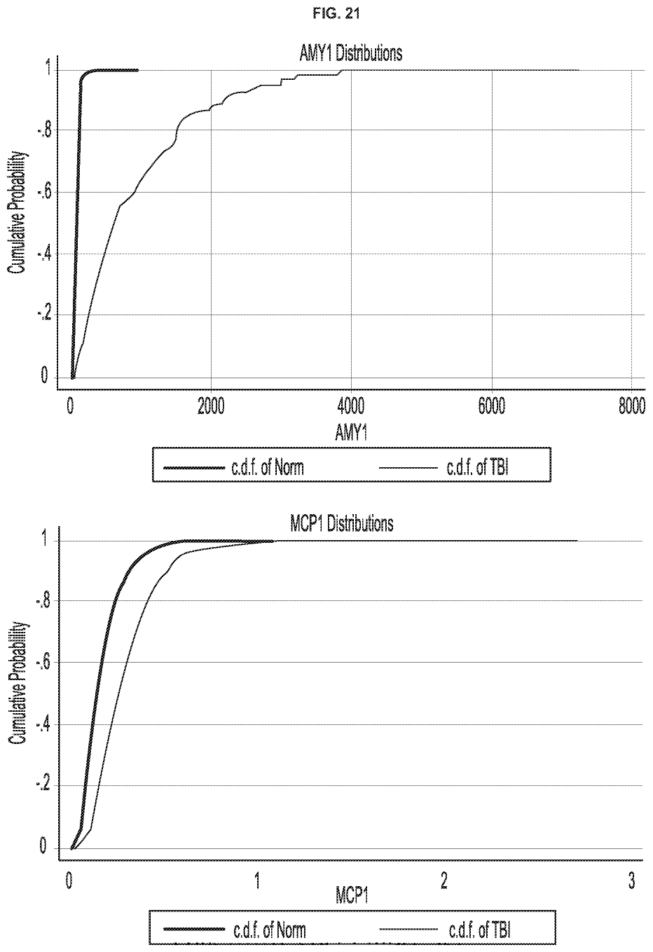

Table 11 summarizes the performance of our classification scheme and three-biomarker panel on all of our human subjects. From the table, we infer an overall sensitivity of 90.4% and a specificity of 92.6% corresponding to a false negative rate (FNR) of 9.6% and a false positive rate (FPR) of 7.4%. The corresponding ROC curve is shown in FIG. 20 and has an AUC of 0.96.

Table 11 indicates classification results obtained from all human samples using the three-biomarker panel. The corresponding sensitivity and specificity are 90.4% and 92.6% respectively.

TABLE 11

| | Condition Negative | Condition Positive |
|---|---|---|
| Predicted Negative | 819 | 16 |
| Predicted Positive | 65 | 151 |
| Total | 884 | 167 |

### CDFs of Human and NHP Data Sets

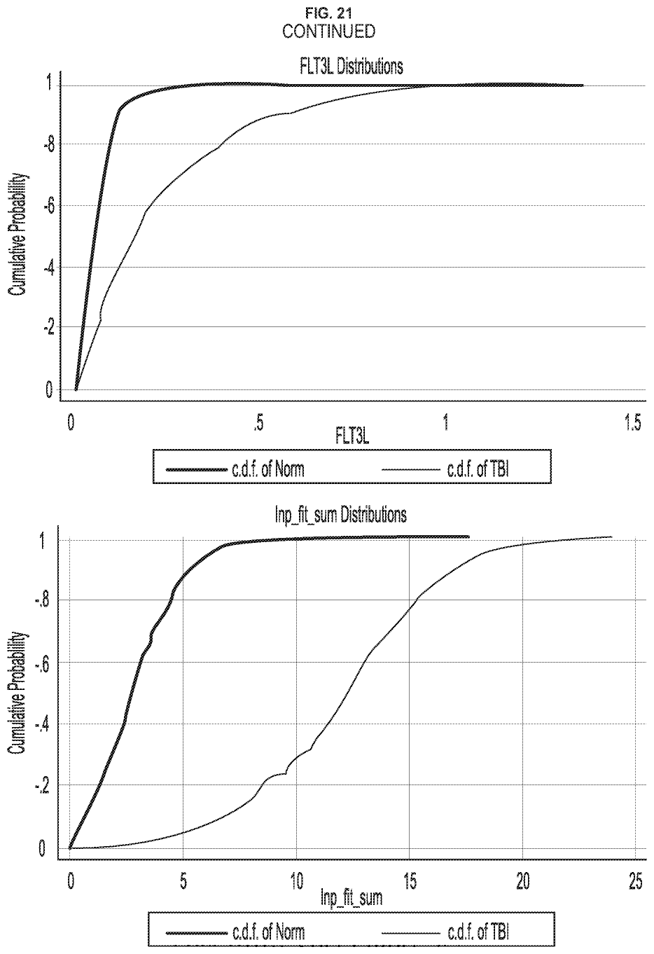

The cumulative distribution function (CDF) is a useful tool for evaluating and comparing the various human and NHP data sets. CDF plots for each protein as well as the composite sum for human normals and TBI patients are shown in FIG. 21. In this figure, it can be seen that for TBI patients, AMY1A exhibits the largest shift to the right from the normal distribution and therefore has the strongest influence. MCP1 exhibits the smallest shift from the normal distribution and therefore has the weakest influence of the three proteins. The CDF plot shows the probability or proportion of observed values of a measured parameter (for example a biomarker concentration or the Principal Component Analysis (PCA) test statistic) that take values less than values shown on the x-axis. For example, in the CDF plot of the protein AMY1, if a value on the x-axis is 500 ng/ml then the height of the curve at x=500 is the probability that the protein is 500 ng/ml or less.

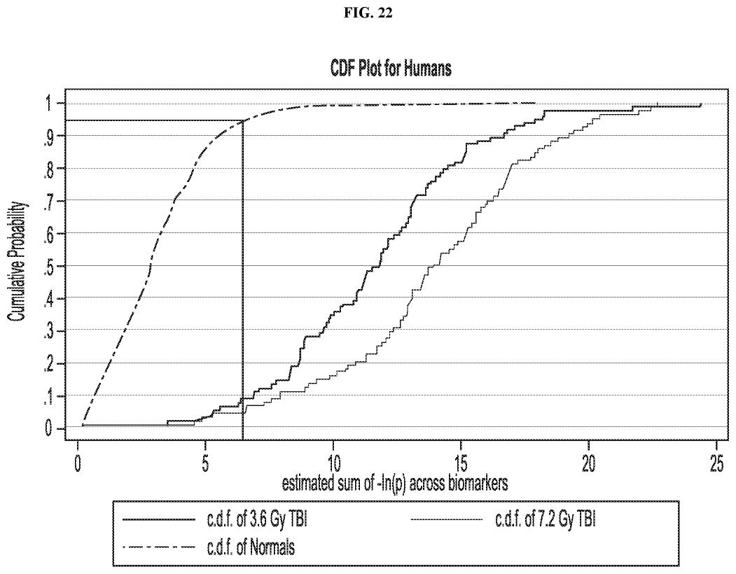

FIG. 22 shows cumulative distribution functions (CDF) of the test statistic (TS) values (sum of −ln(p)) for Human subjects discussed above, with normals and 0, 3.6, and 7.2 Gy fractionated TBI exposure levels, along with the 95% threshold level for the normal CDF plot. As can be seen from the Figure, the CDF plot exhibits increasingly large shifts to the right for higher exposures. Similar trends are seen for the equivalent NHP CDF plots.

### Comparison of Human and NHP CDFs

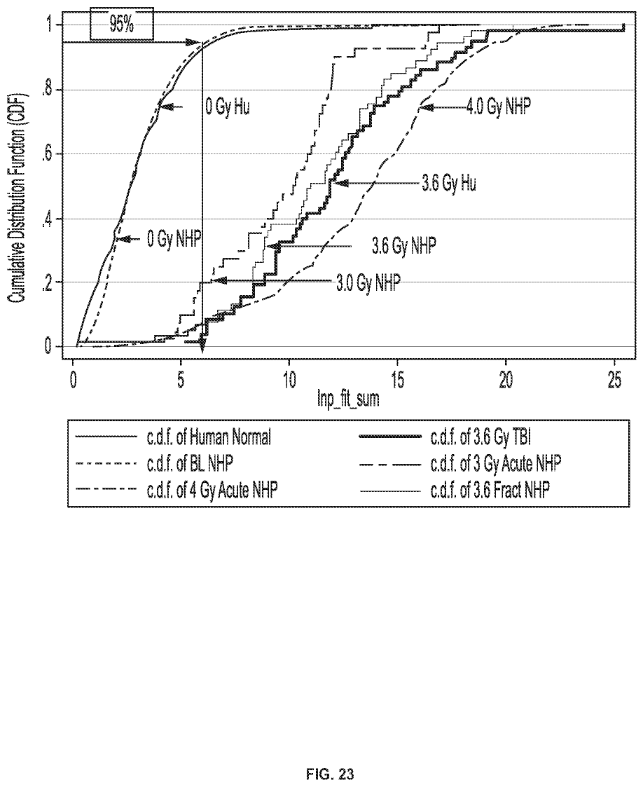

FIG. 23 plots the CDFs for both unexposed humans and NHPs as well as human TBI patients and healthy NHPs receiving a total fractionated dose of 3.6 Gy and healthy NHPs receiving single acute doses of 3 and 4 Gy. In the case of each species, the calculation of the composite biomarker CDF is performed independently. Note that in contrast to the NHP data which covers the 1 to 7 day post exposure time window, this human data is not averaged over a full 1-7 days, because it is based on the specific TBI therapeutic protocol used.

Several features of these CDFs are noteworthy. The CDFs for baseline humans and NHPs are nearly identical demonstrating that using species-normalized data inputs, equivalent results are obtained for each species.

As before, the curves shift to the right with increasing absorbed radiation dose showing excellent discrimination between exposed and unexposed subjects, for both species. The CDFs for 3.6 Gy fractionated NHP and TBI human subjects are nearly identical indicating similar radiation response. The 3 Gy and 4 Gy NHP acute curves nicely bracket the 3.6 Gy curves. We conclude from this that the results obtained from NHPs that receive an acute absorbed dose of ionizing radiation are likely predictive of the response of healthy humans to acute exposure. Finally, based on studies in NHPs, similar radiation responses are observed for our biomarker panel for both acute and fractionated exposures.

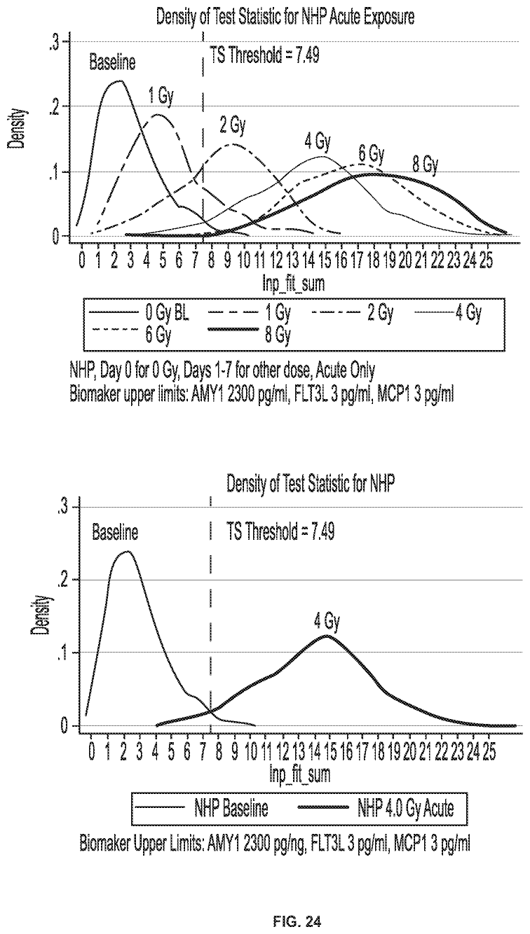

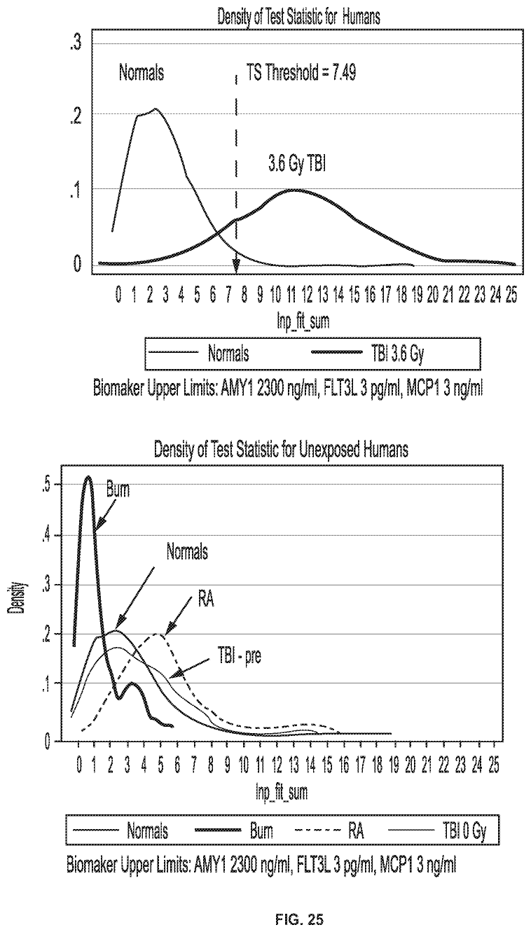

FIG. 24 shows the estimated distribution of test statistic values for NHP exposed to various concentrations (i.e., prior to exposure, and after exposure to 1, 2, 4, 6, or 8 Gy). The curves are density distributions where the total area under each curve is 1.0 (representing 100% of the observations at that exposure level). Density distributions are essentially a smoothed version of a histogram of the observations. The proportion of area under each curve to the left of the TS threshold is the expected proportion of negative classifications and the proportion to the right of the TS threshold is the expected proportion of positive classifications. The TS threshold of 7.49 was selected to yield a very low proportion of positives for unexposed NHP and humans, and a very high proportion of positives for NHP exposed to 4 Gy. If the PCA algorithm was applied to a different set of biomarkers a new threshold may be determined by examining how the percent positive varies across radiation exposure doses and selecting a value that best balances considerations of sensitivity and specificity. FIG. 25 is a simplified version of FIG. 23 that only shows the density distributions for baseline (i.e., pre-exposure) and after exposure to 4 Gy.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
receiving a plurality of human subject biomarker concentration values associated with a human subject, wherein the plurality of human subject biomarkers is associated with a condition;
determining, based on the plurality of human subject biomarker concentration values, a human subject test statistic;
comparing the human subject test statistic to a test statistic threshold, wherein the test statistic threshold is derived based in part on non-human primate (NHP) subject data; and
determining, based on the human subject test statistic exceeding the test statistic threshold, that the human subject has the condition.

2. The method of claim 1, wherein receiving the plurality of subject biomarker concentration values comprises:
measuring an intensity of light reflected from each of a plurality of zones of a lateral flow assay test strip, wherein each of the plurality of human subject biomarkers is associated with one zone of the plurality of zones, and wherein a control is associated with at least one zone of the plurality of zones; and
converting, for each zone of the plurality of zones, the intensity of light into a human subject concentration value for the biomarker of the plurality of human subject biomarkers associated with a respective zone of the plurality of zones.

3. The method of claim 1, wherein the plurality of human subject biomarkers comprise one or more of salivary alpha amylase (AMY1), Flt3 ligand (FLT3L), or monocyte chemotactic protein 1 (MCP1) and wherein the condition is exposure to radiation at 2 Gy or greater.

4. The method of claim 1, wherein the condition is exposure to radiation at 2 Gy or greater.

5. The method of claim 1, wherein determining, based on the plurality of human subject biomarker concentration values, the human subject test statistic comprises determining a sum across the plurality of human subject biomarker concentration values of −ln(Pᵢⱼ*) wherein i denotes a biomarker, and Pᵢⱼ* is an estimate of a probability (Pᵢⱼ) that a person from a reference population would have a biomarker concentration value above the corresponding human subject biomarker concentration value obtained from a j-th sample of the human subject.

6. The method of claim 1, wherein determining, based on the plurality of human subject biomarker concentration values, the human subject test statistic comprises:
for each human subject biomarker concentration value (Cᵢ) of the plurality of human subject biomarker concentration values:
determining a natural log transformation (Lᵢ) by evaluating Lᵢ=ln(Cᵢ);
determining a standardized value (Zᵢ) by evaluating Zᵢ=(Lᵢ−Mᵢ)/Sᵢ, wherein Mᵢ represents a mean value of a natural log of biomarker concentrations in a reference population and wherein Sᵢ represents a standard deviation of the natural log of biomarker concentrations in the reference population;
determining a coefficient Aᵢ and a coefficient Bᵢ, wherein the coefficient Aᵢ comprises a regression coefficient for a constant used to estimate the probability Pᵢ that an observation from a reference population exceeds an observed concentration and wherein the coefficient Bᵢ comprises a regression coefficient for standardized values of −ln(Cᵢ) used to estimate Pᵢ;
estimating a probability (Pᵢ) as Pᵢ*(Zᵢ, Aᵢ, Bᵢ);
determining an inverse natural log transformation of Pᵢ* by evaluating −ln(Pᵢ*); and
determining the human subject test statistic by evaluating Σᵢ(−ln(P*ᵢ)).

7. The method of claim 6, further comprising:
determining if Cᵢ is less than a concentration minimum, wherein if Cᵢ is less than the concentration minimum, setting −ln(Pᵢ*) to a first predefined value;
determining if Cᵢ is greater than a concentration maximum, wherein if Cᵢ is greater than the concentration maximum, setting −ln(Pᵢ*) to a second predefined value; and
determining if −ln(Pᵢ*) is greater than an upper threshold value for acceptable values of −ln(Pᵢ*), wherein if −ln(Pᵢ*) is greater than the upper threshold value, setting −ln(Pᵢ*) to the upper threshold value.

8. The method of claim 7, wherein one or more of the test statistic threshold, Mᵢ, Sᵢ, the coefficient Aᵢ, the coefficient Bᵢ, the concentration minimum, the concentration maximum, or the upper threshold value is received via an RFID tag affixed to a cartridge containing a lateral flow assay test strip.

9. The method of claim 1, further comprising outputting an indication that the human subject has the condition to a display.

10. The method of claim 1, further comprising previously deriving the test statistic threshold based on non-human primate (NHP) subject data such that a False Negative Rate is less than 10% for humans exposed to greater than or equal to 3.6 Gy.

11. An apparatus comprising:
a housing comprising a port for receiving a test strip that supports lateral flow of a fluid sample along a lateral flow direction and comprises a plurality of zones wherein each of a plurality of human subject biomarkers is associated with one zone of the plurality of zones, and wherein a control is associated with at least one zone of the plurality of zones, wherein the plurality of human subject biomarkers are associated with a condition;
a reader configured to obtain separable light intensity measurements from the plurality of zones; and
a data analyzer configured to,
convert, for each zone of the plurality of zones, a light intensity measurement into a human subject concentration value for the biomarker of the plurality of human subject biomarkers associated with a respective zone of the plurality of zones;
determine, based on the plurality of human subject biomarker concentration values, a human subject test statistic;
compare the human subject test statistic to a test statistic threshold, wherein the test statistic threshold is derived based on non-human primate (NHP) subject data; and
determine, based on the human subject test statistic exceeding the test statistic threshold, that the human subject has the condition.

12. The apparatus of claim 11, wherein the plurality of human subject biomarkers comprise one or more of salivary alpha amylase (AMY1), Flt3 ligand (FLT3L), or monocyte chemotactic protein 1 (MCP1).

13. The apparatus of claim 11, wherein the condition is exposure to radiation at 2 Gy or greater.

14. The apparatus of claim 11, wherein the data analyzer is configured to determine, based on the plurality of human subject biomarker concentration values, the human subject test statistic by determining a sum across the plurality of human subject biomarker concentration values of −ln(Pᵢⱼ*) wherein i denotes a biomarker, and Pᵢⱼ* is an estimate of a probability (Pᵢⱼ) that a person from a reference population would have a biomarker concentration value above the corresponding human subject biomarker concentration value obtained from a j-th sample of the human subject.

15. The apparatus of claim 11, wherein the data analyzer is configured to determine, based on the plurality of human subject biomarker concentration values, the human subject test statistic by:
for each human subject biomarker concentration value (Cᵢ) of the plurality of human subject biomarker concentration values:
determining a natural log transformation (Lᵢ) by evaluating L=ln(Cᵢ);
determining a standardized value (Zᵢ) by evaluating Zᵢ=(Lᵢ−Mᵢ)/Sᵢ, wherein Mᵢ represents a mean value of a natural log of biomarker concentrations in a reference population and wherein Sᵢ represents a standard deviation of the natural log of biomarker concentrations in the reference population;
determining a coefficient Aᵢ and a coefficient Bᵢ, wherein the coefficient Aᵢ comprises a regression coefficient for a constant used to estimate the probability Pᵢ that an observation from a reference population exceeds an observed concentration and wherein the coefficient Bᵢ comprises a regression coefficient for standardized values of −ln(Cᵢ) used to estimate Pᵢ;
estimating a probability (Pᵢ) as Pᵢ*(Zᵢ, Aᵢ, Bᵢ);
determining an inverse natural log transformation of Pᵢ* by evaluating −ln(Pᵢ*); and
determining the human subject test statistic by evaluating Σᵢ(−ln(P*ᵢ)).

16. The apparatus of claim 15, further comprising:
determining if Cᵢ is less than a concentration minimum, wherein if Cᵢ is less than the concentration minimum, setting −ln(Pᵢ*) to a first predefined value;
determining if Cᵢ is greater than a concentration maximum, wherein if Cᵢ is greater than the concentration maximum, setting −ln(Pᵢ*) to a second predefined value; and
determining if −ln(Pᵢ*) is greater than an upper threshold value for acceptable values of −ln(Pᵢ*), wherein if −ln(Pᵢ*) is greater than the upper threshold value, setting −ln(Pᵢ*) to the upper threshold value.

17. The apparatus of claim 16, further comprising an RFID reader configured to receive one or more of the test statistic threshold, Mᵢ, Sᵢ, the coefficient Aᵢ, the coefficient Bᵢ, the concentration minimum, the concentration maximum, or the upper threshold value is received via an RFID tag affixed to a cartridge containing the lateral flow assay test strip.

18. The apparatus of claim 11, further comprising a display configured to output an indication that the human subject has the condition.

19. The apparatus of claim 11, further comprising previously deriving the test statistic threshold based on non-human primate (NHP) subject data such that a False Negative Rate is less than 10% for humans exposed to greater than or equal to 3.6 Gy.

20. A computer readable medium with computer-readable instructions for carrying out the method of claim 1.
